(19) United States
(12) Patent Application Publication
(10) Pub. No.: US 2010/0021608 A1
(43) Pub. Date: Jan. 28, 2010

(54) FOOD COMPOSITIONS INCORPORATING STEARIDONIC ACIDS

(75) Inventor: Richard S. Wilkes, Chesterfield, MO (US)

Correspondence Address: Armstrong Teasdale LLP (29135);Christopher M. Goff, One Metropolitan Square, Suite 2600, St. Louis, MO, 63102-2740, US

(73) Assignee: MONSANTO TECHNOLOGY LLC, St. Louis, MO (US)

(21) Appl. No.: 12/521,938

(22) PCT Filed: Jan. 3, 2008
(86) PCT No.: PCT/US08/00052

§ 371 Date: Jul. 1, 2009

Related U.S. Application Data
(60) Provisional application No. 60/878,301, filed on Jan. 3, 2007.

(51) Int. Cl. A23D 9/00; A23D 7/00; A23L 1/30; A23K 1/00; A23K 1/18; A23L 1/36

(52) U.S. Cl. 426/542; 426/601; 426/602; 426/610; 426/654

(57) ABSTRACT

The present invention relates to the improvement of food items through the increased utilization of plant-derived stearidonic acid. Many long chain fatty acids have been classified as being Omega 3 and have been shown to provide several health benefits, including heart health. According to the current invention plant-derived stearidonic acid (18:40)3) has been incorporated into a wide range of food products by using either oil or flour processed from soybeans with enhanced levels of stearidonic acid. These foods range from oil-based products (salad dressing, mayonnaise) to dairy products (milk, cheese) to prepared foods (entrees, side dishes). In addition to improved health benefits the current invention provides food rich in Omega-3 fatty acids that have enhanced storage and/or shelf life characteristics.

### FIELD OF THE INVENTION

The present invention relates to the utilization of transgenically derived stearidonic acid in the development of functional food products. More specifically it relates to an improvement in both the nutritional quality and shelf-life of food products through the use of transgenic plant-derived stearidonic acid.

### BACKGROUND OF THE INVENTION

The present invention is directed to a method for improving foodstuffs through the utilization of plant-derived stearidonic acid (“SDA”). Specifically, the inventor provides techniques and methods for the utilization of plant-derived SDA in foodstuffs that improves nutritional quality. In the past dietary fats have been thought of as valueless or even harmful dietary components. Many studies have made a physiological link between dietary fats and obesity and other pathologies such as atherosclerosis. Given this perception of low nutritional value, consumption of fats has been discouraged by many in the medical establishment.

However, recent studies have determined that despite their relatively simple biological structures there are some types of fats that appear to improve body function in some ways and that may, in fact, be essential to certain physiological processes. The wider class of fat molecules includes fatty acids, isoprenols, steroids, other lipids and oil-soluble vitamins. Among these are the fatty acids. The fatty acids are carboxylic acids, which have from 2 to 26 carbons in their “backbone,” with none, or various numbers of unsaturations in their carbohydrate structure. They generally have dissociation constants (pKa) of about 4.5 indicating that in normal body conditions (physiological pH of 7.4) the vast majority will be in a dissociated form.

With the improvement in nutritional stature for fats and in particular fatty acids, many in the food industry have begun to focus on fatty acids and lipid technology as a new focus for food production. This focus has been particularly intense for the production and incorporation of Omega-3 fatty acids into the diet. Omega-3 fatty acids are long-chain polyunsaturated fatty acids (18-22 carbon atoms in chain length) with the first of the double bonds (“unsaturations”) beginning with the third carbon atom. They are called “polyunsaturated” because their molecules have two or more double bonds “unsaturations” in their carbohydrate chain. They are termed “long-chain” fatty acids since their carbon backbone has at least 18 carbon atoms. In addition to stearidonic acid “SDA” the omega-3 family of fatty acids includes alpha-linolenic acid (“ALA”), eicosapentaenoic acid (“EPA”), and docosahexaenoic acid (“DHA”). ALA is the “base” omega-3 fatty acid, from which EPA and DHA are made in the body through a series of enzymatic reactions, including the production of SDA. Most nutritionists point to DHA and EPA as the most physiologically important of the Omega-3 fatty acids. This synthesis processes from ALA are called “elongation” (the molecule becomes longer by incorporating new carbon atoms) and “desaturation” (new double bonds are created), respectively. In nature, ALA is primarily found in certain plant seeds (e.g., flax) while EPA and DHA mostly occur in the tissues of cold-water predatory fish (e.g., tuna, trout, sardines and salmon), and in some marine algae or microbes that they feed upon.

It is also not widely known that the cold-water marine fish harvested for omega-3's and their use as food do not actually produce the essential omega-3 PUFA's, EPA and DHA. Rather, the long chain PUFA's are biosynthesized by microbes or alga and are passed up the food-chain and collect in the tissues of predatory species. Currently there are two commercially available marine single-cell oils rich in DHA produced by a United States Company—Martek, one from a heterotrophic dinoflagellate (Crypthecodinium cohnii) and the other from a marine thraustochytrid (Schizochytrium sp.). Unfortunately, the cost of production is simply too substantial to commercially justify large scale production and the available supply remains small.

In addition to difficulties with simply securing a supply of Omega-3 fatty acids, are the costs to process omega-3 fatty acids into food products. Even after harvest these costs are also prohibitive to food companies. The reason for additional processing costs is the relative chemical instability of EPA and DHA. These Omega-3 fatty acids can be quickly oxidized leading to undesirable odors and flavors. To reduce the rate of oxidation food processors must therefore either distribute the oil in a frozen condition or encapsulate the desirable fatty acids, each greatly increasing the cost of processing and consequent cost to the consumer. Despite this increased expense—food companies are interested in supplying Omega-3 fatty acids because they believe that health conscious consumers may be willing to pay a small premium for an improved diet if a reliable supply can be developed.

Along with the movement of food companies to develop essential fats and oils as an important component in a healthy diet, governments have begun developing regulations pushing for the adoption of PUFA's in the diet. The difficulty in supplying these needs has been the inability to develop a large enough supply of Omega-3 oil to align with growing marketplace demand. As already mentioned, the Omega-3 fatty acids deemed to be of highest value, EPA and DHA, also chemically degrade very quickly over time limiting commercial access. Importantly, during the rapid process of EPA and DHA oxidation these long chain fatty acids develop rancid or simply unsatisfactory sensory properties that make their inclusion in many foodstuffs difficult or impossible from a commercial acceptance perspective. In addition, with increased demand for Omega-3 fatty acids has come the realization that already depleted global fish stocks cannot meet any significant growth in future human nutritional needs for Omega-3's. These limitations on supply, stability and sourcing greatly increase cost and correspondingly limit the availability of dietary Omega-3's.

Accordingly, a need exists to provide a large-scale stable supply of Omega-3 fatty acids or critical precursors thereto that can be included in food and feed formulations in a commercially acceptable way. The current invention provides this alternative to fish or microbe supplied Omega-3 fatty acids and does so utilizing a comparatively chemically stable Omega-3 fatty acid, SDA as a source that offers neutral taste, cost-effective production and abundant supply as derived from transgenic plants. SDA is the immediate metabolic product of α-linoleic acid (“ALA”), and once in the body is readily metabolized to EPA. The plant species that are specifically included within the group of those that could supply demand are: soybeans, corn, and canola, but also may include other plants as needed. Once produced the SDA of the invention can be used to improve the health characteristics of a great variety of food products. This production can also be scaled-up as needed to both reduce the need to harvest wild fish stocks and to provide essential fatty acid components for aquaculture operations, each easing pressure on global fisheries.

Importantly, the current art suggests that food compositions comprising alpha-linolenic acid are not converted to EPA to any physiologically significant extent when formulated into foods and/or beverages for commercial sale and consumer consumption. The difficulty here is the needed volume of ALA relative to the reasonable volume of consumer foodstuffs. Traditional means of obtaining physiologically relevant amounts of EPA or DHA include the addition of fish oils or algal oils which possess negative attributes of off-flavors and poor stability. In order to contain a concentration of ALA that will lead to a physiologically significant concentration of EPA and DHA in the body, an excessive amount of ALA is required, leading to difficulties in formulating food products and portion sizes that are simply not practicable.

Surprisingly, the inventor has found that the concentration of SDA from the transgenic plant sources of the invention require a far lower concentration in a given food or beverage product to be physiologically significant, these ranges are well within acceptable volume parameters for typical food products. A further benefit is found in the enhanced flavor and stability in comparison with other means of obtaining similar benefits such as direct addition of DHA containing oils like fish oil. As such, the SDA compositions of the invention are uniquely suited fatty acid for both healthy and stable food compositions.

### SUMMARY OF THE INVENTION

The present invention encompasses production of oil from transgenic soybeans engineered to contain significant quantities of stearidonic acid (18:4ω3) for use in food products to improve the health of an end consumer. Sufficient quantities of SDA enriched soybeans have been grown to allow the delivery of soybean oil with a substantial SDA component. This “SDA oil” provides an initial clean flavor, longer shelf-life stability and enhanced nutritional quality relative to either source of Omega-3 oils. The means to maintain oil quality during storage have also been developed. Several food products made from the SDA oil have been produced and found to have similar taste and sensory properties compared to products made from conventional oils, such as soybean oil.

Also according to the current invention, shelf-life testing of food products has also been conducted and the plant-derived SDA oil has substantially improved shelf-life characteristics relative to other Omega-3 containing products. Therefore, a preferred embodiment of the current invention is the usage of the SDA oil produced by transgenic plants in the production of food products for human consumption.

Nutritional studies have shown that, compared to alpha-linolenic acid, SDA is about 5 times more efficiently converted in vivo to EPA. Accordingly, in another embodiment of the current invention plant-derived SDA can be utilized as a neutraceutical supplement or dietary additive for certain pathological conditions.

Specifically, the current invention demonstrates that acceptable food products can be made with stearidonic acid, increasing their shelf-life beyond that of competitive PUFA oils.

Moreover, the method of the current invention also provides for optimizing food formulations to optimize health improvements in end consumers, in the form of an edible oil, processing oil or oil composition, a whole bean extraction for use in a soymilk formulation or as a partial extraction flour-type composition.

In an additional embodiment of the current invention the SDA oils produced by transgenic plants can form the basis for the diet of aquaculture raised fish and/or products from those fish.

In an additional embodiment of the current invention the SDA-oils produced by transgenic plants can form the basis for the diet of beef cattle to improve the nutritional characteristics of beef and/or beef products. Additional embodiments of the current invention may also improve reproductive function.

In an additional embodiment of the current invention the SDA oils produced by transgenic plants can form the basis for the diet of pigs to improve the nutritional characteristics of pork and/or pork products. Additional embodiments of the current invention may also improve reproductive function.

In an additional embodiment of the current invention the SDA oils produced by transgenic plants can form the basis for the diet of chickens to improve the nutritional characteristics of chicken and/or chicken products. Additional embodiments of the current invention may also improve reproductive function.

Other features and advantages of this invention will become apparent in the following detailed description of preferred embodiments of this invention, taken with reference to the accompanying figures.

### BRIEF DESCRIPTION OF THE DRAWINGS

FIG. 1 Shows The Biosynthetic Pathway Of PUFA Metabolism.

FIG. 2 Shows Time Point Testing For Sensory Information For Italian Dressing A-E.

FIG. 3 Shows Time Point Testing For Sensory Information For Ranch Dressing A-E.

FIG. 4 Shows Time Point Testing For Sensory Information For Mayonnaise A-D.

FIG. 5 Shows Time Point Testing For Sensory Information For Soymilk A-B.

FIG. 6 Shows Time Point Testing For Sensory Information For Fruit Smoothies A-C.

FIG. 7 Shows A Graphic Representing The Relative Bioactivity Of Omega-3 Fatty Acids.

FIG. 8 Shows A Process Flow Diagram For The Production Of Soymilk.

FIG. 9 Shows A Process Flow Diagram For The Production Of Vanilla Soymilk.

FIG. 10 Shows A Process Flow Diagram For The Production Of Margarine.

FIG. 11 Shows A Model of Stearidonic Acid.

### DESCRIPTION OF THE PREFERRED EMBODIMENT

The following abbreviations have designated meanings in the specification:

Abbreviation Key:

AA Arachidonic Acid

ALA α-Linolenic Acid

DHA Docosahexanoic Acid

DNA Deoxyribonucleic Acid

EPA Eicosapentanoic Acid

GLA γ-Linolenic Acid

LA Linoleic Acid

mRNA messenger Ribonucleic Acid

PUFA Poly-Unsaturated Fatty Acids

SDA Stearidonic Acid

Explanation of Terms:
Expression—The process of the transcription of a gene to produce the corresponding mRNA and translation of this mRNA to produce the corresponding gene product (i.e., a peptide, polypeptide, or protein).
Feed—Materials available for feeding animals which includes without limitation forage, fodder and concentrates.
Food—Substances which are ingested by humans and contain nutrients which can be metabolized to produce energy.
Gene—Chromosomal DNA, plasmid DNA, cDNA, synthetic DNA, or other DNA that encodes a peptide, polypeptide, protein, or RNA molecule.
Host or Host Organism—Bacteria cells, fungi, animals and animal cells, plants and plant cells, or any plant parts or tissues including protoplasts, calli, roots, tubers, seeds, stems, leaves, seedlings, embryos, and pollen.
Mouthfeel—Means how the substance feels in a human mouth. With regard to taste test profiles this refers to the viscosity, texture and smoothness of the substance being tested.
Nutritional Food Bar—As used herein, the term “Nutritional Food Bar” means a food bar designed to promote health.
Transformation—refers to the introduction of nucleic acid into a recipient host.
Transgene—Any piece of a nucleic acid molecule that is inserted by artifice into a cell, or an ancestor thereof, and becomes part of the genome of the plant or animal which develops from that cell. Such a transgene may include a gene which is partly or entirely exogenous (i.e., foreign) to the transgenic plant or animal, or may represent a gene having identity to an endogenous gene of the plant or animal.
Transgenic—Any cell that includes a nucleic acid molecule that has been inserted by artifice into a cell, or an ancestor thereof, and becomes part of the genome of the plant or animal which develops from that cell.

### DETAILED DESCRIPTION

The present invention relates to a system for an improved method of production of stearidonic acid and its incorporation into the diets of humans and livestock in an effort to improve human health. This production is through the utilization of transgenic plants engineered to produce SDA in high yield to allow commercial incorporation into food products. For the purposes of the current invention the acid and salt forms of fatty acids, for instance, butyric acid and butyrate, arachidonic acid and arachidonate, will be considered interchangeable chemical forms.

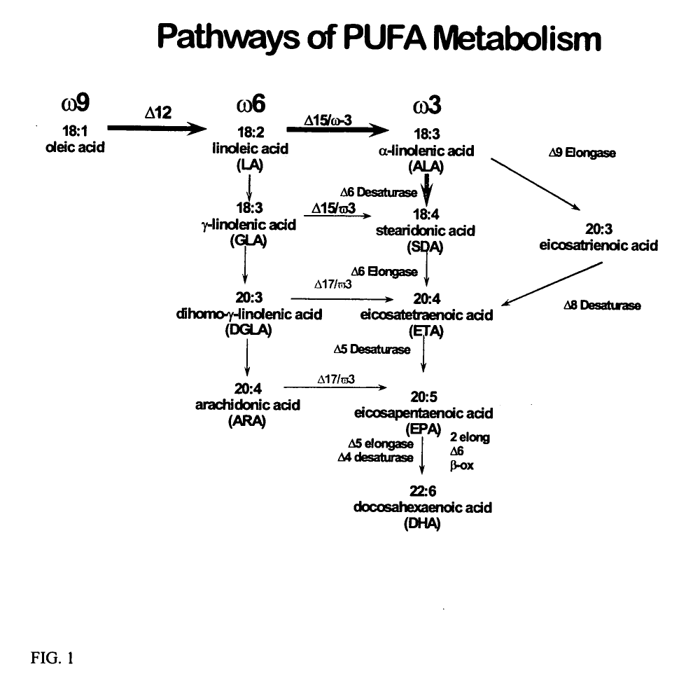

Turning to FIG. 1, all higher plants have the ability to synthesize the main 18 carbon PUFA's, LA and ALA, and in some cases SDA (C18:4n3, SDA), but few are able to further elongate and desaturate these to produce AA, EPA or DHA. Synthesis of EPA and/or DHA in higher plants therefore requires the introduction of several genes encoding all of the biosynthetic enzymes required to convert LA into AA, or ALA into EPA and DHA. Taking into account the importance of PUFAs in human health, the successful production of PUFAs (especially the n-3 class) in transgenic oilseeds, according to the current invention can then provide a sustainable source of these essential fatty acids for dietary use. The “conventional” aerobic pathway which operates in most PUFA-synthesising eukaryotic organisms, starts with A6desaturation of both LA and ALA to yield γ-linolenic (GLA, 18:3n6) and SDA.

### Establishing the Composition of Oils

Turning to Table 1a, it is important to provide a basis of what constitutes ‘normal’ ranges of oil composition vis-à-vis the oil compositions of the current invention. A significant source of data used to establish basic composition criteria for edible oils and fats of major importance has been the Ministry of Agriculture, Fisheries and Food (MAFF) and the Federation of Oils, Seeds and Fats Associations (FOSFA) at the Leatherhead Food Research Association facility in the United Kingdom.

To establish meaningful standards data, it is essential that sufficient samples be collected from representative geographical origins and that the oils be pure. In the MAFF/FOSFA work, over 600 authentic commercial samples of vegetable oilseeds of known origin and history, generally of ten different geographical origins, were studied for each of 11 vegetable oils. The extracted oils were analyzed to determine their overall fatty acid composition (“FAC”). The FAC at the 2-position of the triglyceride, sterol and tocopherol composition, triglyceride carbon number and iodine value, protein values in the oil, melting point and solid fat content as appropriate are determined.

Prior to 1981, FAC data were not included in published standards because data of sufficient quality was not available. In 1981, standards were adopted that included FAC ranges as mandatory compositional criteria. The MAFF/FOSFA work provided the basis for later revisions to these ranges.

In general, as more data became available, it was possible to propose fatty acid ranges much narrower and consequently more specific than those adopted in 1981. Table 1a gives examples of FAC of oils that were adopted by the Codex Alimentarius Commission (CAC) in 1981 and ranges for the same oils proposed at the Codex Committee on Fats and Oils (CCFO) meeting held in 1993.

TABLE 1a
STANDARDS FOR FATTY ACID COMPOSITION OF OILS

| | Soybean oil | | Groundnut oil | | Cottonseed oil | | Sunflower-seed oil | |
|---|---|---|---|---|---|---|---|---|
| Fatty acid | 1981 | 1993 | 1981 | 1993 | 1981 | 1993 | 1981 | 1993 |
| C14:0 | <0.5 | <0.2 | <0.6 | <0.1 | 0.4-2 | 0.6-1 | <0.5 | <0.2 |
| C16:0 | 7-14 | 8-13.3 | 6-16 | 8.3-14 | 17-31 | 21.4-26.4 | 3-10 | 5.6-7.6 |
| C16:1 | <0.5 | <0.2 | <1 | <0.2 | 0.5-2 | 0-1.2 | <1 | <0.3 |
| C18:0 | 1.4-5.5 | 2.4-5.4 | 1.3-6.5 | 1.9-4.4 | 1-4 | 2.1-3.3 | 1-10 | 2.7-6.5 |
| C18:1 | 19-30 | 17.7-26.1 | 35-72 | 36.4-67.1 | 13-44 | 14.7-21.7 | 14-65 | 14-39.4 |
| C18:2 | 44-62 | 49.8-57.1 | 13-45 | 14-43 | 33-59 | 46.7-58.2 | 20-75 | 48.3-74 |
| C18:3 | 4-11 | 5.5-9.5 | <1 | <0.1 | 0.1-2.1 | 0-0.4 | 0-0.7 | 0-0.2 |
| C20:0 | <1 | 0.1-0.6 | 1-3 | 1.1-1.7 | 0-0.7 | 0.2-0.5 | 0-1.5 | 0.2-0.4 |
| C20:1 | <1 | <0.3 | 0.5-2.1 | 0.7-1.7 | 0-0.5 | 0-0.1 | 0-0.5 | 0-0.2 |
| C22:0 | <0.5 | 0.3-0.7 | 1-5 | 2.1-4.4 | 0-0.5 | 0-0.6 | 0-1 | 0.5-1.3 |
| C22:1 | — | <0.3 | <2 | <0.3 | 0-0.5 | 0-0.3 | 0-0.5 | 0-0.2 |
| C22:2 | — | — | — | — | — | — | — | 0-0.3 |
| 024:0 | — | <0.4 | 0.5-3 | 1.1-2.2 | 0-0.5 | 0-0.1 | 0-0.5 | 0.2-0.3 |
| C24:1 | — | — | — | <0.3 | — | — | <0.5 | — |

Sources: CODEX ALIMENTARIUS COMMISSION, 1983 and 1993.

Given the above and according to the current invention, the SDA rich oil produced in an recombinant oilseed plant, provides an oil composition not previously available for food manufacturers. It provides for the incorporation of an Omega-3 oil in food products that was not present in appreciable amounts in typical vegetable oils prior to the current invention. In addition the use of this Omega-3 oil is made possible without the traditional concerns with food sensory qualities, or shelf-life when such oils are delivered from a fish or algal source. After delivery of the oil it can be taken and utilized for the production of baked goods, dairy products, spreads, margarines, sports products, nutrition bars and infant formulas, feed, aquaculture, neutraceutical and medicinal uses. Each having enhanced nutritional content.

Turning to Table 1b, to illustrate the utility of the current invention a variety of food products have been/are being chosen representing a broad range of food categories, to determine the impact of SDA and other Omega-3 oils on product taste and shelf life.

Oxidative stability, as measured by accepted shelf-life sensory tests, is an important PUFA characteristic that determines the useful lifetime and flavor characteristics of fat and oils. Oxidative deterioration in fats and oils can be assessed by wet chemical methods such as peroxide value (PV, which measures peroxides resulting from primary oxidation), and p-anisidine value (AV, which principally measures 2-alkenals resulting from secondary oxidation), or in foods, can be assessed by sensory tasting tests. Selected food categories and products are as follows:

TABLE 1b

| | DAIRY | | PREPARED | OIL BASED | |
|---|---|---|---|---|---|
| BEVERAGES | PRODUCTS | BAKING | FOODS | PRODUCTS | SNACK FOODS |
| Soy milks | Cheeses | Breads | Entrees | Salad | Granola |
| Smoothies | Cream | Rolls | Side Dishes | Dressing | Cereals |
| Fruit Juices | Cheeses | Cakes | Soups | Mayonnaise | Snack/Nutritional |
| Dairy Drinks | Sour Cream | Pastries | Sauces | Margarine/ | Bars |
| | Yogurt | Cookies | Processed | Spreads | Confectionary |
| | Yogurt | Crackers | Meats | Shortening | |
| | Drinks | Muffins | Processed | | |
| | Non Dairy | | Fish | | |
| | Creamers | | Pet Foods | | |
| | Dips | | | | |

According to the current studies the development of food products incorporating transgenic SDA provided several formulations and processes. Additional development and research has been conducted for flavor optimization and the enhancement of shelf-life characteristics. For example, food or beverages that can contain the SDA compositions of the current invention, include baked goods and baked good mixes (e.g., cakes, brownies, muffins, cookies, pastries, pies, and pie crusts), shortening and oil products (e.g., shortenings, margarines, frying oils, cooking and salad oils, popcorn oils, salad dressings, and mayonnaise), foods that are fried in oil (e.g., potato chips, corn chips, tortilla chips, other fried farinaceous snack foods, french fries, doughnuts, and fried chicken), dairy products and artificial dairy products (e.g., butter, ice cream and other fat-containing frozen desserts, yogurt, and cheeses, including natural cheeses, processed cheeses, cream cheese, cottage cheese, cheese foods and cheese spread, milk, cream, sour cream, buttermilk, and coffee creamer), meat products (e.g., hamburgers, hot dogs, wieners, sausages, bologna and other luncheon meats, canned meats, including pasta/meat products, stews, sandwich spreads, and canned fish), meat analogs, tofu, and various kinds of protein spreads, sweet goods and confections (e.g., candies, chocolates, chocolate confections, frostings, and icings, syrups, cream fillings, and fruit fillings), nut butters and various kinds of soups, dips, sauces and gravies. Each of the above examples comprise different embodiments of the current invention.

The current invention bases its formulations on target levels of Omega-3 oils for each food product. These levels were identified based on bio-equivalence of the SDA product. The following information in Table 2a, identifies the targeted Omega 3 levels on a per serving basis:

TABLE 2a

| Omega-3 Source | mg Omega-3 per serving |
|---|---|
| Stearidonic Acid (SDA) | 375 |
| EPA/DHA (fish/algal oil) | 130 |
| ALA (flax oil) | 320 |

Based on this information, preferred formulations of the SDA of the current invention were developed with the appropriate level of stearidonic acid to deliver the targeted levels on a per serving basis. The amount added varied between different applications due to the differences in serving size.

Below are Tables 2b-d reflecting the ranges of the SDA oil compositions of the current invention.

TABLE 2b
SDA Oil Variant-1 (Produced by the Transgenic Plants of the Invention)
ANALYTICAL DATA OF SOYBEAN SEEDS AND OILS - CRUSH, (250 kilograms)

| | SEED | | | CRUDE OIL | | | RBD OIL | | |
|---|---|---|---|---|---|---|---|---|---|
| Moisture (w/w %) | 9.13 | 8.8 | 11.51 | N/A | N/A | N/A | N/A | N/A | N/A |
| Oil content (%) | 19.2 | 18.56 | 19.72 | N/A | N/A | N/A | N/A | N/A | N/A |
| Peroxide value (PV, | N/A | N/A | N/A | 0.46 | 0.00 | 0.06 | 0.0 | 0.0 | 0.0 |
| meq/kg) | | | | | | | | | |
| Free fatty acids (FFA, %) | N/A | N/A | N/A | 0.24 | 0.24 | 0.42 | 0.05 | 0.13 | 0.05 |
| p-Anisidine value (AV) | N/A | N/A | N/A | 0.43 | 0.31 | 0.22 | 0.3 | 0.63 | 0.83 |
| Conjugated dienes (CD) | N/A | N/A | N/A | N/A | N/A | N/A | N/A | N/A | N/A |
| Rancimat @110 C, hrs | N/A | N/A | N/A | N/A | N/A | N/A | 4.6 | 1.89 | 1.85 |
| Trans fatty acids (mg/g) | N/A | N/A | N/A | N/A | N/A | N/A | N/A | N/A | N/A |
| Fatty acid composition | | | | | | | | | |
| (FAC, w/w %) | | | | | | | | | |
| C14:0 (Myristic) | 0.11 | 0.1 | 0.1 | 0.09 | 0.09 | 0.08 | 0.09 | 0.08 | 0.08 |
| C16:0 (Palmitic) | 11.43 | 11.82 | 12.15 | 11.68 | 12.2 | 12 | 11.57 | 11.3 | 12.23 |
| C16:1n7 (Palmitoleic) | 0.1 | 0.09 | 0.09 | 0.1 | 0.12 | 0.14 | 0.1 | 0.09 | 0.14 |
| C18:0 (Stearic) | 4.26 | 4.28 | 4.31 | 4.26 | 4.41 | 4.24 | 4.24 | 4.4 | 4.26 |
| C18:1n9 (Oleic) | 21.09 | 19.44 | 18.54 | 20.88 | 19.28 | 18.6 | 21.16 | 19.3 | 18.74 |
| C18:1 (Octadecenoic) | 1.47 | 1.52 | 1.50 | 1.46 | 1.48 | 1.46 | 1.46 | 1.52 | 1.44 |
| C18:2n6 (Linoleic) | 51.75 | 24.82 | 24.56 | 52.14 | 25.48 | 24.06 | 51.88 | 25.38 | 24.1 |
| C18:3n6 (Gamma | | 5.28 | 6.17 | | 5.23 | 6.15 | | 5.27 | 6.21 |
| linolenic) | | | | | | | | | |
| C18:3n3 (Alpha linolenic) | 8.47 | 10.00 | 10.14 | 8.22 | 10.6 | 10.03 | 8.23 | 10.72 | 10.15 |
| C18:4n3 (Stearidonic) | | 20.40 | 20.90 | | 19.40 | 21.16 | | 20.16 | 21.10 |
| C20:0 (Arachidic) | 0.33 | 0.35 | 0.36 | 0.32 | 0.37 | 0.36 | 0.32 | 0.37 | 0.37 |
| C20:1n9 (Eicosenoic) | 0.16 | 0.17 | 0.18 | 0.15 | 0.24 | 0.24 | 0.15 | 0.18 | 0.22 |
| C20:2n6 (Eicosadienoic) | 0.03 | 0.02 | 0.03 | 0.03 | 0.03 | 0.03 | 0.03 | 0.02 | 0.03 |
| C22:0 (Behenic) | 0.31 | 0.30 | 0.31 | 0.32 | 0.31 | 0.31 | 0.32 | 0.32 | 0.3 |
| C24:0 (Lignoceric) | 0.1 | 0.06 | 0.06 | 0.1 | 0.08 | 0.07 | 0.1 | 0.06 | 0.07 |
| Others | 0.39 | 0.69 | 0.6 | 0.25 | 0.68 | 1.07 | 0.35 | 0.83 | 0.56 |
| Total* | 100.0 | 99.3 | 100.0 | 100.0 | 100.0 | 100.0 | 100.0 | 100.0 | 100.0 |
| Color (5.25″) | N/A | N/A | N/A | 70Y 3.2R | 70Y 3.6R | 70Y 3.8R | 2.8Y 0.1R | 9Y 0.2R | 3.3Y 0.0R |
| | | | | (1″) | (1″) | (1″) | | | |
| Chlorophyll (ppm) | N/A | N/A | N/A | 0.007 | 0.004 | 0.011 | 0.02 | 0.028 | 0.013 |
| Tocopherols (ppm) | | | | | | | | | |
| Alpha | N/A | N/A | N/A | 98.5 | 106 | 101 | 99.4 | 103 | 95.3 |
| Gamma | N/A | N/A | N/A | 940 | 869 | 834 | 914 | 815 | 765 |
| Delta | N/A | N/A | N/A | 305 | 285 | 286 | 293 | 249 | 235 |
| Total | N/A | N/A | N/A | 1343.5 | 1260.0 | 1221.0 | 1306.4 | 1167.0 | 1095.3 |
| Sterols (ppm) | | | | | | | | | |
| Campesterol | N/A | N/A | N/A | 761 | 799 | 677 | 318 | 227 | 588 |
| Stigmasterol | N/A | N/A | N/A | 722 | 684 | 556 | 240 | 130 | 444 |
| Beta-Sitosterol | N/A | N/A | N/A | 1849 | 2196 | 1920 | 1071 | 1021 | 1747 |
| Total | N/A | N/A | N/A | 3332 | 3679 | 3153 | 1629 | 1378 | 2779 |
| Metals (ppm) | | | | | | | | | |
| Phosphorus | N/A | N/A | N/A | 473.6 | 451 | 58.5 | N/A | N/A | N/A |
| Ca | N/A | N/A | N/A | 18.45 | 10.7 | 10.6 | N/A | N/A | N/A |
| Mg | N/A | N/A | N/A | 30.98 | 28.2 | 6.98 | N/A | N/A | N/A |
| Fe | N/A | N/A | N/A | 1.41 | 1.48 | 0.09 | N/A | N/A | N/A |
| Cu | N/A | N/A | N/A | <0.05 | <0.05 | <0.05 | N/A | N/A | N/A |
| Na | N/A | N/A | N/A | 1.75 | 1.39 | <0.20 | N/A | N/A | N/A |

TABLE 2c
SDA Oil Variant-1 (Produced by the Transgenic Plants of the Invention)
ANALYTICAL DATA OF SOYBEAN SEEDS AND OILS - CRUSH,
(5 Metric Tonnes Control Soybeans, 6.8 Tonnes SDA soybeans)

| | Control | | Control | | | Batch 1 | | | | | |
|---|---|---|---|---|---|---|---|---|---|---|---|
| | (NK43 | | (NK43 | SDA | SDA | &2 | Batch | Batch | SDA w | SDA w | SDA |
| | B1) | SDA | B1) | with N2 | no N2 | Combo | 2a | 2b | N2 Batch 1 | N2 Batch 2 | w/o N2 |
| Moisture, %* or ppm | 12.7* | 12.1* | N/A | N/A | N/A | 45.3 | 22.9 | 16.7 | 99.2 | 107.4 | 115.7 |
| Oil content, % | 19.9 | 20.0 | | | | | | | | | |
| Crude fiber, % | 4.43 | 4.55 | N/A | N/A | N/A | N/A | N/A | N/A | N/A | N/A | N/A |
| Ash, % | 4.68 | 4.63 | N/A | N/A | N/A | N/A | N/A | N/A | N/A | N/A | N/A |
| Urease | 2.16 | 2.14 | N/A | N/A | N/A | N/A | N/A | N/A | N/A | N/A | N/A |
| Protein, (N*6.25)% | 36.0 | 36.0 | N/A | N/A | N/A | N/A | N/A | N/A | N/A | N/A | N/A |
| Trypsin inhibitor | 43,300 | 39,000 | N/A | N/A | N/A | N/A | N/A | N/A | N/A | N/A | N/A |
| Free fatty acids (FFA, %) | N/A | N/A | 0.235 | 0.14 | 0.28 | 0.04 | 0.04 | 0.04 | 0.02 | 0.03 | 0.03 |
| Peroxide value (PV, | N/A | N/A | 0.17 | 0.31 | 0.39 | 0.1 | 0.1 | 0.1 | 0.0 | 0.0 | 0.1 |
| meq/kg) | | | | | | | | | | | |
| p-Anisidine value (AV) | N/A | N/A | 0.31 | 0.47 | 0.71 | 2.64 | 0.98 | 0.8 | 0.4 | 1.05 | 1.1 |
| Conjugated dienes (CD) | N/A | N/A | N/A | N/A | N/A | N/A | N/A | N/A | N/A | N/A | N/A |
| Trans fatty acids, % | 0.00 | 0.00 | 0.19 | 0.46 | 0.48 | 0.31 | 0.29 | 0.30 | 0.89 | 0.92 | 0.86 |
| Fatty acid composition | | | | | | | | | | | |
| (FAC, w/w %) | | | | | | | | | | | |
| C14:0 (Myristic) | 0.09 | 0.11 | 0.08 | 0.10 | 0.10 | 0.07 | 0.07 | 0.07 | 0.10 | 0.10 | 0.11 |
| C16:0 (Palmitic) | 11.14 | 12.14 | 10.65 | 12.07 | 12.54 | 10.49 | 10.48 | 10.49 | 12.07 | 12.06 | 12.03 |
| C16:1 (trans- | | | 0.01 | 0.01 | 0.01 | 0.01 | 0.01 | 0.01 | 0.01 | 0.01 | 0.01 |
| Hexadecanoic)** | | | | | | | | | | | |
| C16:1n7 (Palmitoleic) | 0.15 | 0.15 | 0.11 | 0.11 | 0.10 | 0.11 | 0.11 | 0.11 | 0.11 | 0.11 | 0.11 |
| C17:0 (Margaric) | 0.10 | 0.10 | 0.00 | 0.00 | 0.00 | N/A | N/A | N/A | N/A | N/A | N/A |
| C18:0 (Stearic) | 4.38 | 4.19 | 4.65 | 4.19 | 4.26 | 4.66 | 4.64 | 4.64 | 4.19 | 4.19 | 4.19 |
| C18:1 (trans | | | 0.08 | 0.08 | 0.08 | 0.09 | 0.09 | 0.09 | 0.07 | 0.06 | 0.08 |
| Octadecenoic) | | | | | | | | | | | |
| C18:1n9 (Oleic) | 20.40 | 18.35 | 20.64 | 17.92 | 17.91 | 20.70 | 20.66 | 20.68 | 17.92 | 17.92 | 17.96 |
| C18:1 (Octadecenoic) | 1.29 | 1.27 | 1.47 | 1.47 | 1.49 | 1.49 | 1.50 | 1.48 | 1.46 | 1.47 | 1.46 |
| C18:2 (trans- | | | 0.05 | 0.09 | 0.09 | 0.09 | 0.10 | 0.10 | 0.13 | 0.12 | 0.14 |
| Octadecadienoic) | | | | | | | | | | | |
| C18:2n6 (Linoleic) | 53.51 | 35.07 | 53.10 | 35.22 | 35.34 | 53.07 | 53.07 | 53.07 | 35.21 | 35.26 | 35.47 |
| C18:3 (trans- | | | 0.04 | 0.18 | 0.20 | 0.13 | 0.10 | 0.11 | 0.40 | 0.42 | 0.36 |
| Octadecatrienoic) | | | | | | | | | | | |
| C18:3n6 (Gamma | 0.00 | 4.92 | 0.00 | 4.95 | 4.82 | N/A | N/A | N/A | 4.91 | 4.90 | 4.83 |
| linolenic) | | | | | | | | | | | |
| C18:3n3 (Alpha linolenic) | 7.34 | 10.31 | 7.63 | 10.27 | 10.18 | 7.58 | 7.63 | 7.62 | 10.13 | 10.11 | 10.09 |
| C18:4 (trans- | | | 0.00 | 0.11 | 0.10 | N/A | N/A | N/A | 0.28 | 0.31 | 0.27 |
| Octadecatetraenoic) | | | | | | | | | | | |
| C18:4n3 (Stearidonic) | 0.00 | 11.70 | 0.00 | 11.78 | 11.31 | N/A | N/A | N/A | 11.43 | 11.37 | 11.25 |
| C20:0 (Arachidic) | 0.38 | 0.39 | 0.39 | 0.42 | 0.41 | 0.38 | 0.39 | 0.39 | 0.41 | 0.41 | 0.41 |
| C20:1n9 (Eicosenoic) | 0.27 | 0.28 | 0.21 | 0.25 | 0.23 | 0.21 | 0.21 | 0.21 | 0.36 | 0.36 | 0.36 |
| C20:2n6 (Eicosadienoic) | 0.04 | 0.04 | 0.00 | 0.00 | 0.00 | 0.00 | 0.00 | 0.00 | 0.00 | 0.00 | 0.00 |
| C22:0 (Behenic) | 0.38 | 0.33 | 0.40 | 0.33 | 0.34 | 0.41 | 0.40 | 0.40 | 0.35 | 0.35 | 0.36 |
| C24:0 (Lignoceric) | 0.16 | 0.14 | 0.14 | 0.13 | 0.13 | 0.14 | 0.14 | 0.14 | 0.13 | 0.13 | 0.13 |
| Others | 0.39 | 0.53 | 0.35 | 0.32 | 0.34 | 0.38 | 0.39 | 0.38 | 0.35 | 0.35 | 0.37 |
| Total | 100.0 | 100.0 | 100.0 | 100.0 | 100.0 | 100.0 | 100.0 | 100.0 | 100.0 | 100.0 | 100.0 |
| Color (5.25″)** | N/A | N/A | N/A | N/A | N/A | 2.6Y | 1.2Y | 0.9Y | 1.4Y | 6.5Y 0.3R | 3Y |
| | | | | | | 0.2R | 0.0R | 0.0R | 0.0R | | 0.4R |
| Chlorophyll, ppm | N/A | N/A | N/A | N/A | N/A | 0.0 | 0.0 | 0.0 | 0.0 | 0.0 | 0.0 |
| Citric acid, ppm | N/A | N/A | N/A | N/A | N/A | <10 | <10 | <10 | <10 | <10 | <10 |
| Tocopherols (ppm) | | | | | | | | | | | |
| Alpha | N/A | N/A | N/A | N/A | N/A | 90.7 | 84.6 | 87.4 | 151 | 157 | 139 |
| Gamma | N/A | N/A | N/A | N/A | N/A | 727 | 725 | 689 | 683 | 721 | 650 |
| Delta | N/A | N/A | N/A | N/A | N/A | 159 | 171 | 162 | 102 | 104 | 105 |
| Total | N/A | N/A | N/A | N/A | N/A | 976.7 | 980.6 | 938.4 | 936 | 982 | 894 |
| Sterols (ppm) | | | | | | | | | | | |
| campesterol | N/A | N/A | N/A | N/A | N/A | 533 | 459 | 451 | 460 | 495 | 383 |
| stigmasterol | N/A | N/A | N/A | N/A | N/A | 569 | 453 | 448 | 465 | 519 | 364 |
| B-sitosterol | N/A | N/A | N/A | N/A | N/A | 1550 | 1410 | 1380 | 1620 | 1680 | 1480 |
| Other | N/A | N/A | N/A | N/A | N/A | 465 | 398 | 403 | 536 | 581 | 472 |
| Total | N/A | N/A | N/A | N/A | N/A | 3117 | 2720 | 2682 | 3081 | 3275 | 2699 |
| Metals (ppm) | | | | | | | | | | | |
| Phosphorus | N/A | N/A | N/A | N/A | N/A | N/A | N/A | N/A | N/A | N/A | N/A |
| Ca | N/A | N/A | N/A | N/A | N/A | N/A | N/A | N/A | N/A | N/A | N/A |
| Cu | N/A | N/A | N/A | N/A | N/A | N/A | N/A | N/A | N/A | N/A | N/A |
| Fe | N/A | N/A | N/A | N/A | N/A | N/A | N/A | N/A | N/A | N/A | N/A |
| Mg | N/A | N/A | N/A | N/A | N/A | N/A | N/A | N/A | N/A | N/A | N/A |
| Na | N/A | N/A | N/A | N/A | N/A | N/A | N/A | N/A | N/A | N/A | N/A |

TABLE 2d
SDA Oil Variant-1 (Produced by the Transgenic Plants of the Invention)
ANALYTICAL DATA OF SOYBEAN SEEDS AND OILS - CRUSH,
(3 Metric Tonnes Control Soybeans, 5 Tonnes SDA soybeans)

| | | | | | Crude Oil | | RBD Oil | | |
|---|---|---|---|---|---|---|---|---|---|
| | | | SDA Seed | | | Avg. | Avg. | Lt | Hvy |
| | Control Seed | | | SDA | | SDA | Control | Bleach | Bleach- |
| | RR1 | A3525 | MO591 | Comp | Control | Values | Values | SDA | SDA |
| Moisture (w/w % or ppm*) | 11.54 | 10.2 | 10.24 | | | | 33.4* | 38.6* | 55.45* |
| Oil content (%) | 18.90 | 19.59 | 19.28 | 19.08 | | | | | |
| Peroxide value (PV, meq/kg) | 0.3 | 0.46 | 0.5 | 0.5 | 0.21 | 0.26 | 0.0 | 0.0 | 0.0 |
| Free fatty acids (FFA, %) | 0.44 | 0.11 | 0.15 | 0.27 | 0.3 | 0.4 | 0.03 | 0.04 | 0.03 |
| p-Anisidine value (AV) | N/A | N/A | N/A | N/A | 0.34 | 1.63 | 1.07 | 2.35 | 2.05 |
| Conjugated dienes (CD) | N/A | N/A | N/A | N/A | N/A | N/A | N/A | N/A | N/A |
| Trans fatty acids (w/w %) | N/A | N/A | N/A | N/A | 0.19 | 0.48 | 0.32 | 0.63 | 0.67 |
| Fatty acid composition (FAC, | | | | | | | | | |
| w/w %) | | | | | | | | | |
| C14:0 (Myristic) | 0.09 | 0.10 | 0.10 | 0.10 | 0.08 | 0.09 | 0.07 | 0.08 | 0.08 |
| C16:0 (Palmitic) | 10.94 | 11.41 | 11.71 | 12.68 | 11.11 | 12.59 | 10.99 | 12.42 | 12.42 |
| C16:1 (Trans-Hexadecanoic) | N/A | N/A | N/A | 0 | 0.01 | 0.01 | 0.01 | 0.01 | 0.01 |
| C16:1n7 (Palmitoleic) | 0.15 | 0.15 | 0.15 | 0.16 | 0.11 | 0.13 | 0.12 | 0.11 | 0.13 |
| C17:0 (Margaric) | 0.10 | 0.11 | 0.11 | 0.11 | N/A | N/A | 0 | 0 | 0 |
| C18:0 (Stearic) | 4.55 | 4.48 | 4.47 | 4.35 | 4.51 | 4.29 | 4.48 | 4.28 | 4.28 |
| C18:1 (Trans-Octadecenoic) | N/A | N/A | N/A | 0 | 0.08 | 0.08 | 0.08 | 0.07 | 0.06 |
| C18:1n9 (Oleic) | 21.70 | 20.90 | 20.51 | 18.47 | 20.77 | 17.76 | 20.82 | 17.83 | 17.85 |
| C18:1 (Octadecenoic) | 0.96 | 1.14 | 1.09 | 1.11 | 1.51 | 1.58 | 1.49 | 1.56 | 1.57 |
| C18:2 (Trans-Octadecadienoic) | N/A | N/A | N/A | 0 | 0.06 | 0.08 | 0.10 | 0.08 | 0.10 |
| C18:2n6 (Linoleic) | 51.76 | 52.25 | 52.52 | 31.25 | 52.00 | 31.39 | 52.08 | 31.31 | 31.32 |
| C18:3 (Trans-Octadecatrienoic) | N/A | N/A | N/A | 0 | 0.07 | 0.25 | 0.16 | 0.29 | 0.30 |
| C18:3n6 (Gamma linolenic) | 0 | 0.06 | 0 | 5.04 | N/A | 5.10 | 0 | 5.12 | 5.13 |
| C18:3n3 (Alpha linolenic) | 8.29 | 7.91 | 8.03 | 10.50 | 8.15 | 10.48 | 8.09 | 10.41 | 10.38 |
| C18:4 (Trans Octadecatetraenoic) | N/A | N/A | N/A | 0 | N/A | 0.13 | 0 | 0.21 | 0.24 |
| C18:4n3 (Stearidonic) | N/A | 0.16 | N/A | 14.59 | N/A | 14.64 | 0 | 14.77 | 14.68 |
| C20:0 (Arachidic) | 0.39 | 0.36 | 0.37 | 0.40 | 0.38 | 0.38 | 0.37 | 0.38 | 0.38 |
| C20:1n9 (Eicosenoic) | 0.26 | 0.25 | 0.24 | 0.29 | 0.24 | 0.26 | 0.22 | 0.27 | 0.28 |
| C20:2n6 (Eicosadienoic) | 0.04 | 0.04 | 0.04 | 0.03 | 0.04 | 0.03 | 0.04 | 0.04 | 0.05 |
| C22:0 (Behenic) | 0.41 | 0.34 | 0.34 | 0.33 | 0.38 | 0.32 | 0.37 | 0.34 | 0.34 |
| C24:0 (Lignoceric) | 0.14 | 0.13 | 0.12 | 0.11 | 0.13 | 0.09 | 0.13 | 0.10 | 0.10 |
| Others | 0.21 | 0.22 | 0.20 | 0.49 | 0.39 | 0.33 | 0.39 | 0.31 | 0.31 |
| Total | 100.0 | 100.0 | 100.0 | 100.0 | 100.0 | 100.0 | 100.0 | 100.0 | 100.0 |
| Color (5.25″) | N/A | N/A | N/A | N/A | 70Y 2.9R | 70Y 3.7R | 5.2Y | 5.5Y | 4.3Y 0.3R |
| | | | | | (1″) | (1″) | 0.4R | 0.3R | |
| Chlorophyll (ppm) | N/A | N/A | N/A | N/A | 0.156 | 0.033 | 0.0 | 0.0 | 0.0 |
| Citric acid (ppm) | N/A | N/A | N/A | N/A | N/A | N/A | <10 | <10 | <10 |
| Tocopherols (ppm) | | | | | | | | | |
| Alpha | N/A | N/A | N/A | N/A | 96.1 | 111 | 87.6 | 106 | 94.9 |
| Gamma | N/A | N/A | N/A | N/A | 830 | 860 | 723 | 777 | 738 |
| Delta | N/A | N/A | N/A | N/A | 238 | 221 | 183 | 176 | 163 |
| Total | N/A | N/A | N/A | N/A | 1164.1 | 1192 | 993.6 | 1059 | 995.9 |
| Sterols (ppm) | | | | | | | | | |
| Campesterol | N/A | N/A | N/A | N/A | 778 | 668 | 674 | 532 | 498 |
| Stigmasterol | N/A | N/A | N/A | N/A | 773 | 673 | 656 | 512 | 476 |
| Beta-Sitosterol | N/A | N/A | N/A | N/A | 1860 | 1880 | 1700 | 1640 | 1570 |
| Others | N/A | N/A | N/A | N/A | 577 | 732 | 498 | 623 | 599 |
| Total | N/A | N/A | N/A | N/A | 3988 | 3953 | 3528 | 3307 | 3143 |
| Metals (ppm) | | | | | | | | | |
| Phosphorus | N/A | N/A | N/A | N/A | 330 | 756 | N/A | N/A | N/A |
| Ca | N/A | N/A | N/A | N/A | 18.6 | 52.8 | N/A | N/A | N/A |
| Mg | N/A | N/A | N/A | N/A | 23.6 | 47 | N/A | N/A | N/A |
| Fe | N/A | N/A | N/A | N/A | 0.67 | 0.59 | N/A | N/A | N/A |
| Cu | N/A | N/A | N/A | N/A | <0.05 | <0.05 | N/A | N/A | N/A |
| Na | N/A | N/A | N/A | N/A | <0.20 | <0.20 | N/A | N/A | N/A |

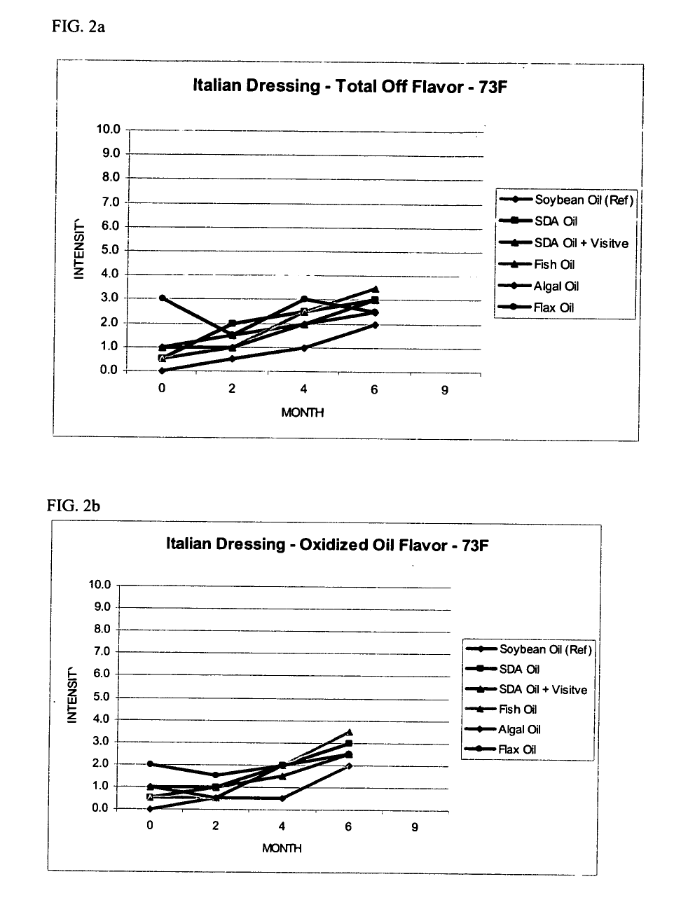

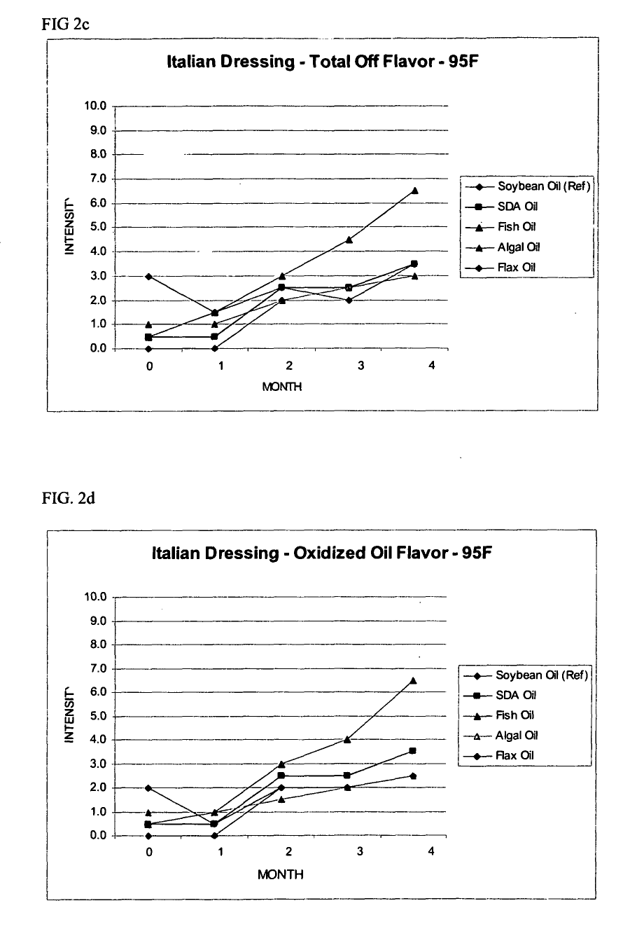

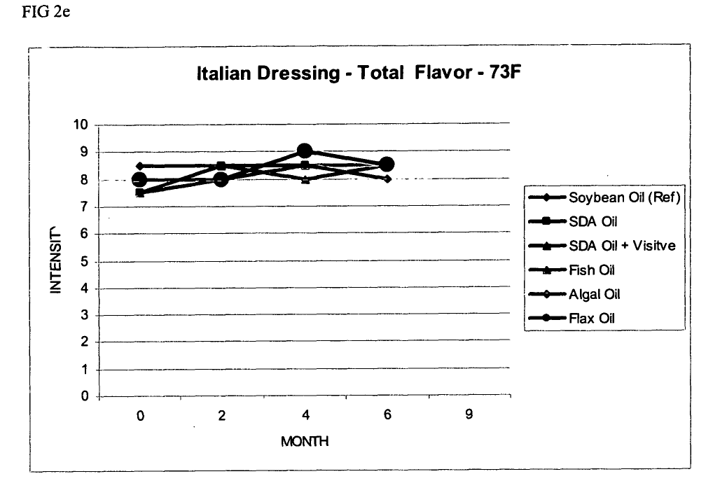

For the instant invention the primary source of stearidonic acid was oil extracted from transgenic soybeans which have been engineered to produce high levels of stearidonic acid. The soybeans were processed at an oil processing facility and oil was extracted consistent with the methods described in US Patent Applications 2006/0111578, and 2006/0111254. In addition to oil, flour was made from the transgenic and control soybeans typical of industry practices in processing full-fatted soy flour. One example of a food formulation utilizing the SDA of the invention is found in Table 3a-3c, and FIGS. 2a-2e below. General attributes of Italian style dressings according to preferred embodiments of the current invention are provided in Tables 4a-4c.

TABLE 3a
Italian Salad Dressing - Shelf Life Attributes -

| | Soybean Oil (reference) | | | | | | | |
|---|---|---|---|---|---|---|---|---|
| | | 95° F. | 95° F. | 95° F. | 95° F. | 73° F. | 73° F. | 73° F. |
| | Initial | 1 mo | 2 mo | 3 mo | 4 mo | 2 mo | 4 mo | 6 mo |
| APPEARANCE | | | | | | | | |
| Opacity | 5 | 5 | 5 | 5 | 5 | 5 | 5 | 5 |
| Color | 5 | 5 | 6 | 6 | 6 | 5 | 5 | 5 |
| AROMA | | 55 | | | | | | |
| Total Aroma | 7.5 | 7.5 | 7.5 | 8 | 8.5 | 7.5 | 7.5 | 7.5 |
| Vinegar | 6 | 6 | 5.5 | 6 | 5.5 | 6 | 6 | 5.5 |
| Pungent | 5 | 5 | 5 | 5.5 | 5.5 | 5 | 4.5 | 5 |
| Total Onion/ | 4 | 4.5 | 3.5 | 3.5 | 3 | 4.5 | 4 | 4 |
| Garlic/Herb | | | | | | | | |
| Total Oil | 2 | 2.5 | 3 | 3.5 | 3.5 | 2.5 | 2.5 | 2.5 |
| Total Off | 0 | 0 | 2 | 2.5 | 3 | 0.5 | 1 | 1.5 |
| Oxidized Oil | 0 | 0 | 1.5 | 2 | 2.5 | 0.5 | 0.5 | 1.5 |
| FLAVOR | | | | | | | | |
| Total Flavor | 8.5 | 8 | 8.5 | 9 | 9 | 8.5 | 8.5 | 8 |
| Vinegar | 6 | 6 | 6 | 6.5 | 6 | 6 | 6 | 5.5 |
| Pungent | 6 | 6 | 6 | 6.5 | 6 | 6 | 6 | 5.5 |
| Total Onion/ | 5 | 5 | 4.5 | 4 | 3.5 | 5.5 | 4.5 | 4.5 |
| Garlic/Herb | | | | | | | | |
| Sour | 6 | 6 | 6 | 6.5 | 7 | 6.5 | 6.5 | 6 |
| Salty | 6.5 | 7 | 6.5 | 6.5 | 7 | 6.5 | 7 | 7 |
| Total Oil | 3 | 3 | 4 | 4 | 4 | 3.5 | 3.5 | 3 |
| Total Off | 0 | 0 | 2 | 2.5 | 3.5 | 0.5 | 1 | 2 |
| Oxidized Oil | 0 | 0 | 2 | 2 | 2.5 | 0.5 | 0.5 | 2 |
| TEXTURE | | | | | | | | |
| Viscosity by Mouth | 4 | 4 | 4.5 | 4.5 | 4 | 4 | 4 | 4 |
| Oily Mouthfeel (after 5 | 7 | 7 | 7.5 | 7.5 | 7.5 | 7 | 7.5 | 7 |
| seconds) | | | | | | | | |
| Comments: | | very | slight | oxidized oil, | oxidized | very | slightly | |
| | | similar | cardboard, | old herb, | oil, | slight | oxidized | |
| | | to | slight | slightly | cardboard | oxidized | oil, slightly | |
| | | control | pondy, | waxy | cooked oil | oil | cardboard | |
| | | | slight | | | | | |
| | | | painty | | | | | |

| | SDA Oil | | | | | | | |
|---|---|---|---|---|---|---|---|---|
| | | 95° F. | 95° F. | 95° F. | 95° F. | 73° F. | 73° F. | 73° F. |
| | Ini | 1 mo | 2 mo | 3 mo | 4 mo | 2 mo | 4 mo | 6 mo |
| APPEARANCE | | | | | | | | |
| Opacity | 6 | 5.5 | 5.5 | 5 | 6 | 5.5 | 6 | 7 |
| Color | 5 | 5 | 7 | 6 | 6.5 | 5 | 5 | 6 |
| AROMA | | | | | | | | |
| Total Aroma | 7 | 8 | 8.5 | 8 | 8 | 7 | 7.5 | 7.5 |
| Vinegar | 5.5 | 6 | 5.5 | 6 | 5.5 | 5.5 | 6 | 5.5 |
| Pungent | 5 | 5 | 5 | 5 | 5 | 4.5 | 5 | 4.5 |
| Total Onion/ | 4 | 4 | 3.5 | 3.5 | 3 | 3.5 | 3.5 | 4 |
| Garlic/Herb | | | | | | | | |
| Total Oil | 2.5 | 2.5 | 3.5 | 3 | 3.5 | 2.5 | 2.5 | 2.5 |
| Total Off | 0.5 | 0.5 | 2.5 | 2.5 | 3 | 1 | 2 | 2 |
| Oxidized Oil | 0.5 | 0.5 | 2.5 | 2.5 | 2.5 | 0.5 | 1.5 | 2 |
| FLAVOR | | | | | | | | |
| Total Flavor | 7.5 | 8 | 9 | 9 | 9 | 8.5 | 8.5 | 8.5 |
| Vinegar | 5.5 | 5.5 | 6 | 6.5 | 6 | 6 | 6 | 6 |
| Pungent | 5.5 | 5.5 | 6 | 6.5 | 6 | 5.5 | 6 | 6 |
| Total Onion/ | 5 | 4.5 | 4 | 4 | 3.5 | 4.5 | 4.5 | 4.5 |
| Garlic/Herb | | | | | | | | |
| Sour | 6 | 6 | 5.5 | 6.5 | 7 | 5.5 | 6 | 6.5 |
| Salty | 6.5 | 6.5 | 6.5 | 7 | 7 | 6.5 | 7 | 6.5 |
| Total Oil | 3.5 | 3.5 | 4 | 3.5 | 4 | 3.5 | 3.5 | 3.5 |
| Total Off | 0.5 | 0.5 | 2.5 | 2.5 | 3.5 | 2 | 2.5 | 3 |
| Oxidized Oil | 0.5 | 0.5 | 2.5 | 2.5 | 3 | 1 | 2 | 3 |
| TEXTURE | | | | | | | | |
| Viscosity by Mouth | 4.5 | 4 | 4 | 4 | 4 | 4.5 | 4 | 4 |
| Oily Mouthfeel (after 5 | 7 | 7 | 7 | 7.5 | 7.5 | 7 | 7 | 7 |
| seconds) | | | | | | | | |
| Comments: | very | very | pondy, | slightly | pondy, | slight | slightly | |
| | slight | slight | slight | pondy, | painty, | pondy, | pondy, | |
| | oxidized | cardboard | beany, | slightly | slightly | slight | old oil | |
| | oil and | | very | fishy, | fishy | beany | | |
| | very | | slight | oxidized | | | | |
| | slight | | fishy | oil | | | | |
| | beany/ | | | | | | | |
| | pondy | | | | | | | |

Scale range = 0 to 15

TABLE 3b
Italian Salad Dressing - Shelf Life Attributes

| | | 95° F. | 95° F. | 95° F. | 95° F. | 73° F. | 73° F. | 73° F. |
|---|---|---|---|---|---|---|---|---|
| | Ini | 1 mo | 2 mo | 3 mo | 4 mo | 2 mo | 4 mo | 6 mo |
| | Fish Oil | | | | | | | |
| APPEARANCE | | | | | | | | |
| Opacity | 6.5 | 5 | 5 | 5 | 5 | 6 | 6 | 6 |
| Color | 5 | 5 | 5.5 | 6 | 7.5 | 5 | 5 | 5 |
| AROMA | | | | | | | | |
| Total Aroma | 6.5 | 7.5 | 8.5 | 9 | 9 | 7 | 7 | 7 |
| Vinegar | 5.5 | 6 | 5.5 | 5.5 | 5 | 5.5 | 5.5 | 5.5 |
| Pungent | 4.5 | 4.5 | 5 | 4.5 | 5 | 4.5 | 4.5 | 5 |
| Onion/ | 3.5 | 3 | 3.5 | 3 | 3 | 3.5 | 3.5 | 3.5 |
| Garlic/Herb | | | | | | | | |
| Total Oil | 3 | 3 | 3.5 | 5 | 6 | 2.5 | 2.5 | 3 |
| Total Off | 0.5 | 1 | 3.5 | 5 | 6 | 1 | 2 | 3 |
| Oxidized Oil | 0.5 | 1 | 3 | 4.5 | 5.5 | 0.5 | 1.5 | 3 |
| FLAVOR | | | | | | | | |
| Total Flavor | 7.5 | 7.5 | 9 | 9.5 | 10 | 8 | 8.5 | 8.5 |
| Vinegar | 5.5 | 6 | 6 | 5.5 | 5 | 6 | 6.5 | 6 |
| Pungent | 5 | 6 | 6 | 6 | 5 | 6 | 6.5 | 5.5 |
| Total Onion/ | 4.5 | 4.5 | 4 | 3.5 | 3.5 | 5.5 | 4 | 4 |
| Garlic/Herb | | | | | | | | |
| Sour | 5.5 | 6 | 6 | 6 | 7 | 6 | 6.5 | 6 |
| Salty | 6.5 | 6.5 | 7 | 6.5 | 7 | 7 | 6.5 | 7 |
| Total Oil | 4 | 3.5 | 4 | 5 | 6.5 | 3.5 | 4 | 3.5 |
| Total Off | 0.5 | 1.5 | 3 | 4.5 | 6.5 | 1 | 2.5 | 3.5 |
| Oxidized Oil | 0.5 | 1 | 3 | 4 | 6 | 0.5 | 2 | 3.5 |
| TEXTURE | | | | | | | | |
| Viscosity by | 5 | 4.5 | 4.5 | 4.5 | 4 | 4.5 | 4 | 4 |
| Mouth | | | | | | | | |
| Oily | 8 | 8 | 7.5 | 7.5 | 7.5 | 8 | 7 | 7 |
| Mouthfeel | | | | | | | | |
| (after 5 | | | | | | | | |
| seconds) | | | | | | | | |
| Comments: | very | slight | pondy, | distinctly | strong | very | slightly | waxy, |
| | slight | oxidized | cardboard, | fishy | fishy | slight | fishy, | cardboard |
| | oxidized | oil, | heavy | | | oxidized | slightly | |
| | oil aroma | sight | oil, | | | oil | pondy, | |
| | and | beany | slight | | | | slightly | |
| | flavor | | painty | | | | motor | |
| | | | | | | | oil | |
| | Algal Oil | | | | | | | |
| APPEARANCE | | | | | | | | |
| Opacity | 5.5 | 5 | 5 | 5 | 5.5 | 5.5 | 5.5 | 6 |
| Color | 5 | 5 | 5.5 | 6 | 7 | 5 | 5 | 4.5 |
| AROMA | | | | | | | | |
| Total Aroma | 7 | 7.5 | 7.5 | 8 | 8 | 7 | 7.5 | 7 |
| Vinegar | 5.5 | 6 | 5.5 | 6 | 5 | 5.5 | 5.5 | 5.5 |
| Pungent | 5 | 5.5 | 4.5 | 5 | 4.5 | 5 | 5 | 4.5 |
| Onion/ | 3.5 | 3.5 | 3.5 | 3 | 3 | 3.5 | 3.5 | 3.5 |
| Garlic/Herb | | | | | | | | |
| Total Oil | 3 | 2.5 | 3 | 3 | 3.5 | 2.5 | 3 | 2.5 |
| Total Off | 1 | 1 | 2 | 2 | 3 | 1 | 2 | 2 |
| Oxidized Oil | 1 | 1 | 1.5 | 1.5 | 2.5 | 1 | 1.5 | 2 |
| FLAVOR | | | | | | | | |
| Total Flavor | 7.5 | 7.5 | 8.5 | 8.5 | 9 | 8 | 8.5 | 8 |
| Vinegar | 5.5 | 6 | 6 | 6 | 6 | 6 | 6.5 | 5.5 |
| Pungent | 5.5 | 6 | 6 | 6 | 6 | 6 | 6 | 5.5 |
| Onion/ | 4.5 | 4.5 | 4.5 | 4 | 3 | 4.5 | 4.5 | 4.5 |
| Garlic/Herb | | | | | | | | |
| Sour | 6 | 6 | 6 | 6.5 | 7 | 6 | 6.5 | 5.5 |
| Salty | 6.5 | 6.5 | 6.5 | 6.5 | 7 | 6.5 | 7 | 6.5 |
| Total Oil | 4 | 3.5 | 3.5 | 4 | 4 | 3.5 | 3.5 | 3.5 |
| Total Off | 1 | 1 | 2 | 2.5 | 3 | 1 | 2 | 2.5 |
| Oxidized Oil | 1 | 1 | 1.5 | 2 | 2.5 | 0.5 | 2 | 2.5 |
| TEXTURE | | | | | | | | |
| Viscosity by | 5 | 4 | 4 | 4 | 4 | 4.5 | 4 | 4.5 |
| Mouth | | | | | | | | |
| Oily Mouthfeel | 7.5 | 7 | 7 | 7 | 7 | 7.5 | 7 | 7 |
| (after 5 seconds) | | | | | | | | |
| Comments: | slight | slight | slight | pondy, | pondy, | slight | slightly | slightly |
| | oxidized | oxidized | cardboard, | heavy oil, | slightly | oxidized | oxidized | cardboard |
| | oil aroma | oil, slight | slight | reheated | rubbery, | oil, slight | oil, | slightly |
| | and | cardboard | oxidized oil | oil | oxidized | cardboard, | slightly | painty |
| | flavor, | | | | heavy oil | slight | reheated | |
| | very | | | | | heated oil | heavy oil | |
| | slight | | | | | | | |
| | pondy | | | | | | | |

TABLE 3c
Italian Salad Dressing - Shelf Life Attributes

| | Flax Oil | | | | | | | |
|---|---|---|---|---|---|---|---|---|
| | | 95° F. | 95° F. | 95° F. | 95° F. | 73° F. | 73° F. | 73° F. |
| | Ini | 1 mo | 2 mo | 3 mo | 4 mo | 2 mo | 4 mo | 6 mo |
| APPEARANCE | | | | | | | | |
| Opacity | 5.5 | 5 | 5 | 6 | 5.5 | 5.5 | 5 | 5.5 |
| Color | 5 | 5 | 5.5 | 6 | 7 | 5 | 5 | 5 |
| AROMA | | | | | | | | |
| Total Aroma | 7 | 7 | 7.5 | 8 | 8 | 7 | 7 | 7 |
| Vinegar | 5.5 | 6 | 6 | 6 | 6 | 6 | 5.5 | 5.5 |
| Pungent | 5 | 5 | 5 | 5.5 | 5.5 | 4.5 | 4 | 5 |
| Total Onion/ | 3.5 | 4 | 3.5 | 3 | 3 | 3.5 | 4 | 3.5 |
| Garlic/Herb | | | | | | | | |
| Total Oil | 3.5 | 3 | 3 | 3 | 3.5 | 3 | 3 | 3 |
| Total Off | 2 | 1.5 | 2.5 | 2.5 | 3 | 1.5 | 2.5 | 2.5 |
| Oxidized Oil | 1.5 | 1 | 2.5 | 2 | 2.5 | 1 | 1.5 | 2 |
| FLAVOR | | | | | | | | |
| Total Flavor | 8 | 8 | 8.5 | 9 | 9 | 8 | 9 | 8.5 |
| Vinegar | 6 | 5.5 | 6 | 6.5 | 6 | 6 | 6 | 5.5 |
| Pungent | 5.5 | 5.5 | 6 | 6 | 6 | 6 | 6 | 5.5 |
| Total Onion/ | 4 | 5 | 4.5 | 4 | 3.5 | 5 | 5 | 4.5 |
| Garlic/Herb | | | | | | | | |
| Sour | 6 | 5.5 | 6 | 6.5 | 6.5 | 5.5 | 6.5 | 5.5 |
| Salty | 6.5 | 6.5 | 6.5 | 6.5 | 7 | 6.5 | 7 | 6.5 |
| Total Oil | 4 | 4 | 4 | 3.5 | 4 | 4 | 4 | 3.5 |
| Total Off | 3 | 1.5 | 2.5 | 2 | 3.5 | 1.5 | 3 | 2.5 |
| Oxidized Oil | 2 | 0.5 | 2 | 2 | 2.5 | 1.5 | 2 | 2.5 |
| TEXTURE | | | | | | | | |
| Viscosity by | 5 | 4.5 | 4.5 | 4 | 4 | 5 | 4.5 | 4 |
| Mouth | | | | | | | | |
| Oily Mouthfeel | 8 | 7.5 | 7.5 | 7.5 | 7 | 7.5 | 7.5 | 7 |
| (after 5 | | | | | | | | |
| seconds) | | | | | | | | |

TABLE 4a
ITALIAN SALAD DRESSING
SDA SALAD DRESSING FORMULATIONS - ITALIAN

| | Variant | | | | |
|---|---|---|---|---|---|
| | Control | SDA | Fish Oil | Algal Oil | Flax Oil |
| | Formula Number | | | | |
| | 50-RA-325-000 | | | | |
| INGREDIENT | % | 50-RA-326-000 | 50-RA-328-000 | 50-RA-330-000 | 50-RA-327-000 |
| Liquid Soybean Oil | 44.5000 | 33.1700 | 43.0700 | 43.2700 | 42.9700 |
| Omega 3 Oil | | 11.33 | 1.43 | 1.23 | 1.53 |
| Water | 39.3530 | 39.3530 | 39.3530 | 39.3530 | 39.3530 |
| Egg Yolk, Liquid, 10% Salt | 2.9000 | 2.9000 | 2.9000 | 2.9000 | 2.9000 |
| Viegar, White Distilled, 120 gr | 2.8500 | 2.8500 | 2.8500 | 2.8500 | 2.8500 |
| Sugar, White, Fine Granulated | 2.5000 | 2.5000 | 2.5000 | 2.5000 | 2.5000 |
| Buttermilk Powder, Cultured LOL#20631 | 2.1000 | 2.1000 | 2.1000 | 2.1000 | 2.1000 |
| Salt, Regular, Non Iodized | 1.7000 | 1.7000 | 1.7000 | 1.7000 | 1.7000 |
| Flavor, Cultured Buttermilk, Cargill#24521 | 1.5000 | 1.5000 | 1.5000 | 1.5000 | 1.5000 |
| Garlic, Dehydrated, Granular | 0.4500 | 0.4500 | 0.4500 | 0.4500 | 0.4500 |
| Onion, Dehydrated, Granular | 0.4400 | 0.4400 | 0.4400 | 0.4400 | 0.4400 |
| Mustard Flour, Wisconsin Spice SP448 | 0.4000 | 0.4000 | 0.4000 | 0.4000 | 0.4000 |
| Acid, Phosphoric, 75% | 0.4000 | 0.4000 | 0.4000 | 0.4000 | 0.4000 |
| Gum, Xanthan, 60 mesh, Regular | 0.2750 | 0.2750 | 0.2750 | 0.2750 | 0.2750 |
| Preservative, Potassium Sorbate | 0.2000 | 0.2000 | 0.2000 | 0.2000 | 0.2000 |
| Monosodium Glutamate (MSG) | 0.2000 | 0.2000 | 0.2000 | 0.2000 | 0.2000 |
| Preservative, Sodium Benzoate, Granular | 0.1000 | 0.1000 | 0.1000 | 0.1000 | 0.1000 |
| Pepper, Black, 30-60 mesh | 0.1000 | 0.1000 | 0.1000 | 0.1000 | 0.1000 |
| Parsley, Dehydrated, Granular −10 +30 | 0.0250 | 0.0250 | 0.0250 | 0.0250 | 0.0250 |
| Preservative, EDTA, Calcium Disodium | 0.0070 | 0.0070 | 0.0070 | 0.0070 | 0.0070 |
| TOTAL | 100.0000 | 100.0000 | 100.0000 | 100.0000 | 100.0000 |

TABLE 4b
ITALIAN SALAD DRESSING
Italian Salad Dressing Production Process:

| | |
|---|---|
| 1. | Check that the mixer is in good working condition, free and clear |
| | of dust & dirt, sealed tight, mill set correctly. |
| 2. | Set mix tank speed to 25 hz. |
| 3. | Meter in water to mix tank. |
| 4. | Add preservatives (Benzoate, Sorbate, EDTA) into mix tank. |
| 5. | Make gum slurry (Xanthan Gum + 400 g soybean oil) |
| 6. | Add to Dixie tank, mix for 3 minutes |
| 7. | Add the rest of the dry ingredients to the Dixie mill. |
| 8. | Adjust mix tank speed to 45 hz. |
| 9. | Add HFCS, caramel color, and Yellow No. 6 to the Dixie tank |
| 10. | Slowly add remainder of soybean oil and if appropriate, Omega 3 oil |
| 11. | Add distilled vinegar, mix for 30 seconds |
| 12. | Open mix tank valve, and set pump speed to 30 hz. |
| 13. | Turn on pum to pack; colloid mill is off. |
| 14. | Pack into bulk or individual containers, cap. |

TABLE 4c
ITALIAN SALAD DRESSING
SHELF LIFE PRODUCTION
ANALTYICAL/MICRO RESULTS
ITALIAN DRESSING

| | Control | SDA | Fish Oil | Algal Oil | Flax Oil |
|---|---|---|---|---|---|
| | 50-RA-252- | 50-RA-248- | 50-RA-264- | 50-RA-266- | 50-RA-265- |
| | 000 | 000 | 000 | 000 | 000 |
| pH | 3.51 | 3.52 | 3.53 | 3.52 | 3.51 |
| Total Acidity | 1.01 | 1.02 | 1.00 | 1.01 | 1.02 |
| Total Solids | 2.56 | 2.51 | 2.50 | 2.52 | 2.53 |
| Bostwick (viscosity) | 18.9 cm | 19.1 cm | 19.25 cm | 19.0 cm | 18.9 cm |
| Total Plate Count | <10 | <10 | <10 | <10 | <10 |
| Lactics | <10 | <10 | <10 | <10 | <10 |
| Yeast | <10 | <10 | <10 | <10 | <10 |
| Mold | <10 | <10 | <10 | <10 | <10 |

According to the methods of the current invention samples of various salad dressings were submitted to a contracting food laboratory for confirmatory studies and analysis of various embodiments of the invention. The general approach to the shelf-life testing is for 5 attribute panelists to taste the dressings and come to consensus regarding the attributes and intensity (on a 15 pt scale—0 being absent, 15 being extreme) for each dressing. The lists of attributes identified by the panelists are in the attached documents. Additional attributes are identified as warranted. The characteristics of attribute testing are provided below, Table 5, along with the data from sensory testing at various time points, Table 6.

TABLE 5
SDA DRESSING DEFINITIONS OF SENSORY ATTRIBUTES

| | |
|---|---|
| APPEARANCE | |
| Yellow Color | The intensity of the yellow color in the sample, from light to dark |
| | yellow. |
| AROMA/FLAVOR | |
| Total Aroma | The total aroma intensity of the sample. |
| Total Flavor | The total flavor intensity of the sample, including the basic tastes. |
| Total Oil | The intensity of aroma/flavor of any type of oil, including oxidized oil. |
| Oxidized Oil | The intensity of aroma/flavor of oxidized oil, described as old oil that |
| | has undergone oxidation, characterized as cardboard, beany, painty, or |
| | fishy. |
| Total Off Aroma/Flavor | The intensity of aroma/flavor of believed to not intended in the |
| | product, includes oxidized oil and other off notes. The nature of the |
| | off note is to be described. |
| Mayonnaise/Dairy | The intensity of the aroma/flavor associated with mayonnaise or dairy |
| | product. |
| Vinegar | The intensity of the aroma/flavor of white vinegar or acetic acid. |
| Onion/Garlic/Herb | The intensity of aroma/flavor associated with onion, garlic, and all |
| | dried and fresh green herbs. |
| Sour | One of the four basic tastes, perceived primarily on the sides of the |
| | tongue; common to acids. |
| Salty | One of the four basic tastes, perceived primarily on the sides of the |
| | tongue; common to sodium chloride (table salt). |
| FEELING FACTORS | |
| Pungent | The amount of burning or irritation of the nasal cavity produced by |
| | smelling the sample, such as with horseradish. |
| TEXTURE | |
| Viscosity by Mouth | The degree of thickness of the sample as perceived when manipulated |
| | in the mouth. |
| Oily Mouthcoating | The amount of coating perceived on the soft tissues of the mouth |
| AFTERTASTE | |
| Total Aftertaste | The total aftertaste intensity of the sample. |

### Example 1

### Salad Dressing

The tables above represent the data developed for a preferred embodiment of the current invention. Please also see FIGS. 2a-2e for graphical representation of the data out to four months. According to the data provided herein, the samples containing SDA are significantly less off-flavored than corresponding fish and algal Omega-3 oil formulations, providing the benefit of the presence of an omega-3 formulation without the substantially shortened shelf-life and limited stability. Due to pungent flavors and extremely unpleasant odors the fish and algal derived oils simply could not be tested and were removed from the 3 months accelerated evaluation period whereas the SDA composition of the invention was not. Overall the SDA compositions of the invention demonstrate improved stability, reduced degradation and consequent enhanced shelf-life for commercial utilization in conjunction with the delivery of beneficial Omega-3's into the diet.

With regard to specific salad dressing embodiments the SDA compositions of the invention developed utilized for enhanced Ranch Dressings maintained their flavor profile longer that the fish and algal oils after 6 months room temperature storage. For Italian dressings, the more complex flavor system does do some masking, but the SDA containing dressings of the current invention are again less off flavored than comparable based fish/algal dressings.

### Italian Salad Dressings:

According to the current invention the shelf-life studies, at room temperature and accelerated studies, were completed through 4 months. Each sample has been evaluated by the trained attribute panel in a food laboratory at 0, 2 and 4 months at room temperature and at 1, 2 and 3 months accelerated temperature (95° F.). For Ranch Dressings, the fish and algal oil samples were only smelled at 3 months due to high off flavor and character at the two month point. All other samples, including those containing the SDA oil of the invention, were evaluated at 3 months. This is typical for accelerated shelf life evaluations.

According to the methods of the current invention the Italian dressings have demonstrated significant stability in terms of flavor relative to other omega-3 containing test subjects. Accelerated testing has been completed through four months testing at 95° F. At this point, all of the products exhibited off flavors, with the fish oils demonstrating the highest in off notes. Significantly, the SDA formulations of the invention were similar to the soybean oil reference.

According to the methods of the current invention the Ranch-style dressings demonstrated significant improvements according to sensory parameters relative to Fish Oil and Algal Oil formulations containing other Omega-3's. Also according to the invention, accelerated testing has been completed. High intensity off flavors developed in the fish and algal samples at two months whereas the SDA oil of the invention and the reference soybean oil could be evaluated according to sensory parameters at 3 months. The reference and flax samples exhibited more characteristic flavors and less off flavor than the SDA oil of the invention. The SDA oil of the invention exhibited more characteristic flavors and less off flavors than the fish and algal samples. This demonstrates that SDA has improved shelf life vs. fish and algal oils. In addition, room temperature testing was completed for the formulations according to the current invention through 4 months. Results indicate that the SDA samples of the invention indicate that the SDA product of the invention has a significantly lower profile for off flavors and unpleasant odors relative to other omega-3 sources, including fish and algal oils.

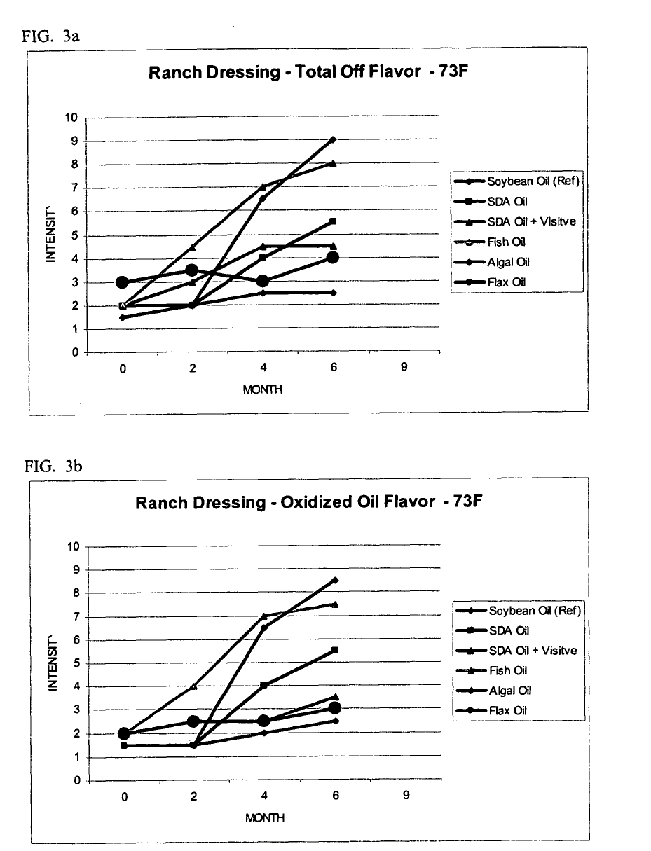

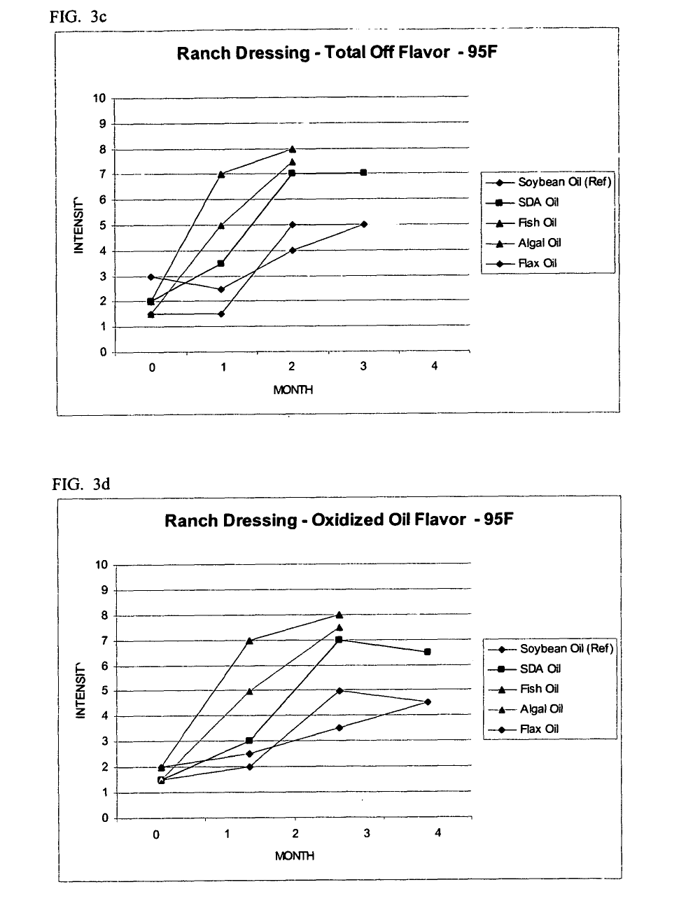

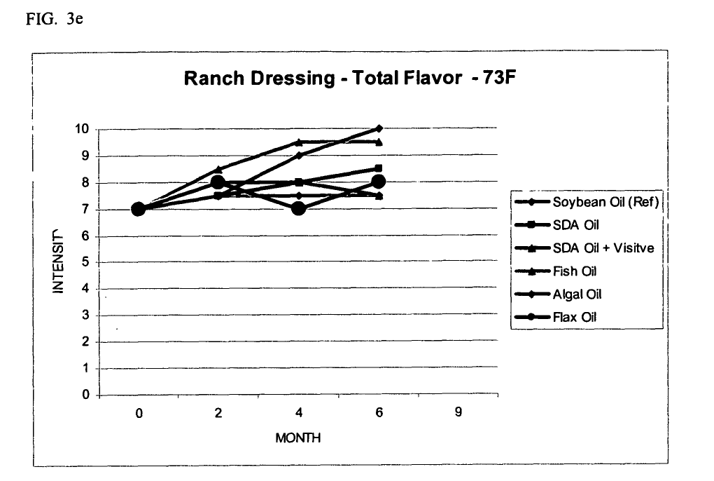

The data for both Italian and Ranch type dressings and charts that demonstrate the characteristics for the evaluation are attached in Tables 1-11 and FIGS. 2 and 3.

### Example 2

### Ranch Salad Dressing

TABLE 6
Ranch Salad
Dressing Shelf Life Attributes

| | | 95° F. | 95° F. | 95° F. | 73° F. | 73° F. | | 95° F. | 95° F. | 95° F. | 73° F. | 73° F. |
|---|---|---|---|---|---|---|---|---|---|---|---|---|
| | Ini | 1 mo | 2 mo | 3 mo | 2 mo | 4 mo | Ini | 1 mo | 2 mo | 3 mo | 2 mo | 4 mo |
| | Soybean Oil (reference) | | | | | | SDA Oil | | | | | |
| APPEARANCE | | | | | | | | | | | | |
| Yellow Color | 4 | 5 | 5 | 6 | 4 | 4 | 4 | 4.5 | 5 | 6 | 4 | 4 |
| AROMA | | | | | | | | | | | | |
| Total Aroma | 6.5 | 6.5 | 6.5 | 7.5 | 6.5 | 7 | 6.5 | 7 | 8 | 8.5 | 6.5 | 7 |
| Mayonnaise | 4 | 4 | 3.5 | 3 | 4 | 3.5 | 4 | 4 | 2.5 | 1.5 | 4 | 3 |
| Dairy/Cultured | 2.5 | 2.5 | 2 | 1.5 | 2 | 2 | 2.5 | 2 | 1.5 | 1 | 2.5 | 1.5 |
| Dairy | | | | | | | | | | | | |
| Vinegar | 4 | 4 | 3.5 | 3 | 3.5 | 3.5 | 3.5 | 3.5 | 2.5 | 2.5 | 3.5 | 3 |
| Pungent | 4 | 4 | 4 | 3.5 | 3.5 | 4 | 3.5 | 3.5 | 5 | 4.5 | 4 | 4 |
| Total Onion/ | 3 | 3 | 2 | 2 | 2.5 | 2.5 | 2.5 | 2.5 | 1.5 | 1 | 2.5 | 2 |
| Garlic/Herb | | | | | | | | | | | | |
| Total Oil | 2.5 | 2.5 | 4 | 4.5 | 3 | 3 | 3 | 3 | 5.5 | 6 | 3 | 3.5 |
| Total Off | 1 | 1 | 4 | 4.5 | 2 | 2 | 1.5 | 3 | 5.5 | 6.5 | 1.5 | 3 |
| Oxidized Oil | 1 | 1 | 3.5 | 4 | 1.5 | 1.5 | 1 | 3 | 5.5 | 6 | 1 | 3 |
| FLAVOR | | | | | | | | | | | | |
| Total Flavor | 7 | 7.5 | 8 | 8.5 | 7.5 | 7.5 | 7 | 7.5 | 8.5 | 9 | 7.5 | 8 |
| Mayonnaise | 5 | 5.5 | 3.5 | 3.5 | 5 | 4 | 5 | 5 | 3 | 2.5 | 4.5 | 3.5 |
| Dairy/Cultured | 3 | 3 | 2 | 2 | 2.5 | 2.5 | 3 | 2 | 1.5 | 1.5 | 2.5 | 2 |
| Dairy | | | | | | | | | | | | |
| Vinegar | 4 | 4 | 3.5 | 3.5 | 3.5 | 4 | 3.5 | 4 | 2.5 | 3.5 | 4 | 3.5 |
| Pungent | 4 | 4 | 4.5 | 4 | 4 | 4 | 3.5 | 4 | 5 | 5 | 4 | 4.5 |
| Total Onion/ | 4 | 4 | 2.5 | 2 | 3 | 3.5 | 3.5 | 3 | 2 | 2 | 3.5 | 3 |
| Garlic/Herb | | | | | | | | | | | | |
| Sour | 4.5 | 4.5 | 5 | 5 | 4.5 | 4.5 | 4 | 4 | 5 | 5.5 | 4.5 | 5 |
| Total Oil | 3.5 | 3.5 | 5 | 4.5 | 4.5 | 3.5 | 4 | 4 | 7 | 6.5 | 4 | 4.5 |
| Total Off | 1.5 | 2 | 5 | 5 | 2 | 2.5 | 2 | 3.5 | 7 | 7 | 2 | 4 |
| Oxidized Oil | 1.5 | 2 | 5 | 4.5 | 1.5 | 2 | 1.5 | 3 | 7 | 6.5 | 1.5 | 4 |
| TEXTURE | | | | | | | | | | | | |
| Viscosity by | 6 | 6 | 6 | 6 | 6 | 6 | 6 | 6.5 | 6 | 6 | 6 | 6 |
| Mouth | | | | | | | | | | | | |
| Oily Mouthfeel | 5 | 5.5 | 5 | 5 | 5 | 5 | 5.5 | 6 | 5 | 5 | 5 | 5 |
| (after 5 sec) | | | | | | | | | | | | |
| Comments: | | very | cardboard, | oxidized | slight | slightly | slight | pondy, | primarily | fishy, | slight | fishy, |
| | | slight | slight | oil, musty | oxidized | oxi- | oxidized, | fishy | pondy, | pondy, | oxidized | painty, |
| | | oxi- | oxidized | (sweat | oil, slight | dized | slight | | fishy, | oxidized | oil | SO2 |
| | | dized | oil | socks) | cardboard | oil | beany | | linseed | oil-painty | | |
| | | oil | | | | | | | oil | | | |
| | Fish Oil | | | | | | Algal Oil | | | | | |
| APPEARANCE | | | | | | | | | | | | |
| Yellow Color | 4 | 4.5 | 5 | 6.5 | 4 | 4 | 5 | 5.5 | 5.5 | 6 | 5 | 4.5 |
| AROMA | | | | | | | | | | | | |
| Total Aroma | 6.5 | 8.5 | 9 | 10.5 | 8 | 8.5 | 6.5 | 7.5 | 8.5 | 10 | 6 | 8 |
| Mayonnaise | 4 | 2 | 2 | 0.5 | 3.5 | 2 | 4 | 3 | 2.5 | 0.5 | 3.5 | 2 |
| Dairy/Cultured | 2.5 | 1 | 1 | 0.5 | 2 | 1.5 | 2 | 2 | 1 | 0.5 | 2 | 1.5 |
| Dairy | | | | | | | | | | | | |
| Vinegar | 4 | 2 | 2 | 2 | 3 | 2.5 | 3.5 | 3 | 2 | 2 | 3 | 2.5 |
| Pungent | 4 | 2.5 | 5.5 | 5.5 | 4 | 4.5 | 3.5 | 3 | 5 | 5 | 3.5 | 4.5 |
| Total Onion/ | 3 | 1.5 | 1 | 0.5 | 2 | 1.5 | 3 | 2 | 1 | 1 | 2 | 1.5 |
| Garlic/Herb | | | | | | | | | | | | |
| Total Oil | 2.5 | 6 | 6.5 | 8.5 | 4 | 5.5 | 2.5 | 5 | 6 | 7.5 | 3.5 | 4.5 |
| Total Off | 1 | 6.5 | 7 | 9.5 | 4 | 5 | 1 | 4 | 6 | 8.5 | 2 | 4.5 |
| Oxidized Oil | 1 | 6.5 | 6.5 | 8.5 | 3.5 | 5 | 1 | 4 | 6 | 7.5 | 1.5 | 4.5 |
| FLAVOR | | | | | | | | | | | | |
| Total Flavor | 7 | 9 | 9.5 | | 8.5 | 9.5 | 7 | 8 | 9 | | 7.5 | 9 |
| Mayonnaise | 5 | 2 | 2.5 | | 4.5 | 2 | 5 | 3.5 | 2.5 | | 4.5 | 2 |
| Dairy/Cultured | 3 | 1.5 | 1 | | 2 | 1 | 3 | 2 | 1.5 | | 2 | 1.5 |
| Dairy | | | | | | | | | | | | |
| Vinegar | 4 | 2 | 2 | | 3.5 | 2.5 | 3.5 | 3.5 | 2 | | 3.5 | 3 |
| Pungent | 4 | 2.5 | 6 | | 4 | 5 | 4 | 3.5 | 6 | | 3.5 | 4.5 |
| Total Onion/ | 4 | 1 | 1.5 | | 2.5 | 1.5 | 3.5 | 3 | 1.5 | | 2.5 | 1.5 |
| Garlic/Herb | | | | | | | | | | | | |
| Sour | 4.5 | 3.5 | 5.5 | | 5 | 5 | 4 | 3.5 | 5.5 | | 4 | 5 |
| Total Oil | 4 | 7 | 7.5 | | 5 | 7.5 | 3.5 | 5.5 | 7.5 | | 4.5 | 6.5 |
| Total Off | 2 | 7 | 8 | | 4.5 | 7 | 1.5 | 5 | 7.5 | | 2 | 6.5 |
| Oxidized Oil | 2 | 7 | 8 | | 4 | 7 | 1.5 | 5 | 7.5 | | 1.5 | 6.5 |
| TEXTURE | | | | | | | | | | | | |
| Viscosity by | 6 | 6 | 6 | | 6 | 6 | 6.5 | 6.5 | 6.5 | | 6 | 6 |
| Mouth | | | | | | | | | | | | |
| Oily Mouthfeel | 5.5 | 5 | 5 | | 5 | 5 | 5 | 6 | 5 | | 5.5 | 5 |
| (after 5 sec) | | | | | | | | | | | | |
| Comments: | slight | strong | strong | fishy | fishy, | strong | very | fishy | strong | fishy, | oxidized | fishy, |
| | beany, | fishy, | fishy | | pondy, | fish | slight | | fishy, | pondy | oil, slight | pondy |
| | slight | slight | | | old | | oxidized | | pondy | | pondy, | |
| | oxi- | pondy | | | vegetables | | oil | | | | slight | |
| | dized | | | | | | | | | | cardboard | |
| | oil | | | | | | | | | | | |

Flax Oil

| | | 95° F. | 95° F. | 95° F. | 73° F. | 73° F. |
|---|---|---|---|---|---|---|
| | Ini | 1 mo | 2 mo | 3 mo | 2 mo | 4 mo |
| APPEARANCE | | | | | | |
| Yellow Color | 4.5 | 5 | 5.5 | 6 | 5 | 4.5 |
| AROMA | | | | | | |
| Total Aroma | 6 | 7 | 6.5 | 8 | 6.5 | 6 |
| Mayonnaise | 3.5 | 4.5 | 3.5 | 3 | 4 | 3 |
| Dairy/Cultured Dairy | 3 | 2.5 | 1.5 | 1.5 | 2 | 2 |
| Vinegar | 3.5 | 4 | 3 | 3 | 3 | 3.5 |
| Pungent | 3.5 | 4 | 4 | 3.5 | 3.5 | 3.5 |
| Total Onion/Garlic/Herb | 3 | 3 | 1.5 | 2 | 2.5 | 2 |
| Total Oil | 3 | 3 | 4 | 4 | 3 | 3 |
| Total Off | 2 | 2 | 3.5 | 4.5 | 2 | 2 |
| Oxidized Oil | 1.5 | 1.5 | 3.5 | 4 | 1.5 | 2 |
| FLAVOR | | | | | | |
| Total Flavor | 7 | 7 | 7.5 | 8.5 | 8 | 7 |
| Mayonnaise | 4.5 | 5 | 3.5 | 3.5 | 5 | 4 |
| Dairy/Cultured Dairy | 3 | 3 | 2 | 2 | 2.5 | 2.5 |
| Vinegar | 3.5 | 4 | 3 | 3.5 | 3.5 | 4 |
| Pungent | 4 | 3.5 | 4.5 | 4 | 4 | 4.5 |
| Total Onion/Garlic/Herb | 3.5 | 3.5 | 2.5 | 2.5 | 3 | 2.5 |
| Sour | 4.5 | 4 | 5 | 5 | 5 | 5 |
| Total Oil | 4 | 4 | 4.5 | 5 | 4.5 | 4 |
| Total Off | 3 | 2.5 | 4 | 5 | 3.5 | 3 |
| Oxidized Oil | 2 | 2.5 | 3.5 | 4.5 | 2.5 | 2.5 |
| TEXTURE | | | | | | |
| Viscosity by Mouth | 6.5 | 6.5 | 6 | 6 | 6 | 6 |
| Oily Mouthfeel (after 5 sec) | 6 | 5.5 | 5 | 5 | 5.5 | 5 |
| Comments: | slight | slight | pondy, | musty | pondy, | cardboard, |
| | fishy | oxidized | beany, | (sweat | slightly | slightly old |
| | | oil, slight | oxidized | socks), | sour | parmesean, |
| | | fishy | oil | oxidized | milk | slightly |
| | | | | oil, | | pondy |
| | | | | slightly | | |
| | | | | fishy, | | |
| | | | | pondy | | |

Scale = 0 1o 15
Note:
color indicates variance from reference soy oil at initial timepoint; yellow = +/−1.0, orange = +/−1.5 to 2.0, red = /<2.5

TABLE 7a
SDA SALAD DRESSING FORMULATIONS - RANCH

| | Variant | | | | |
|---|---|---|---|---|---|
| | Control | SDA | Fish Oil | Algal Oil | Flax Oil |
| | Formula Number | | | | |
| | 50-RA-325- | | | | |
| | 000 | 50-RA-326- | 50-RA-328- | 50-RA-330- | 50-RA-327- |
| INGREDIENT | % | 000 | 000 | 000 | 000 |
| Liquid Soybean Oil | 44.5000 | 33.1700 | 43.0700 | 43.2700 | 42.9700 |
| Omega 3 Oil | | 11.33 | 1.43 | 1.23 | 1.53 |
| Water | 39.3530 | 39.3530 | 39.3530 | 39.3530 | 39.3530 |
| Egg Yolk, Liquid, 10% Salt | 2.9000 | 2.9000 | 2.9000 | 2.9000 | 2.9000 |
| Vinegar, White Distilled, 120 gr | 2.8500 | 2.8500 | 2.8500 | 2.8500 | 2.8500 |
| Sugar, White, Fine Granulated | 2.5000 | 2.5000 | 2.5000 | 2.5000 | 2.5000 |
| Buttermilk Powder, Cultured LOL#20631 | 2.1000 | 2.1000 | 2.1000 | 2.1000 | 2.1000 |
| Salt, Regular, Non Iodized | 1.7000 | 1.7000 | 1.7000 | 1.7000 | 1.7000 |
| Flavor, Cultured Buttermilk, | 1.5000 | 1.5000 | 1.5000 | 1.5000 | 1.5000 |
| Cargill#24521 | | | | | |
| Garlic, Dehydrated, Granular | 0.4500 | 0.4500 | 0.4500 | 0.4500 | 0.4500 |
| Oniion, Dehydrated, Granular | 0.4400 | 0.4400 | 0.4400 | 0.4400 | 0.4400 |
| Mustard Flour, Wisconsin Spice SP448 | 0.4000 | 0.4000 | 0.4000 | 0.4000 | 0.4000 |
| Acid, Phosphoric, 75% | 0.4000 | 0.4000 | 0.4000 | 0.4000 | 0.4000 |
| Gum, Xanthan, 60 mesh, Regular | 0.2750 | 0.2750 | 0.2750 | 0.2750 | 0.2750 |
| Preservative, Potassium Sorbate | 0.2000 | 0.2000 | 0.2000 | 0.2000 | 0.2000 |
| Monosodium Glutamate (MSG) | 0.2000 | 0.2000 | 0.2000 | 0.2000 | 0.2000 |
| Preservative, Sodium Benzoate, | 0.1000 | 0.1000 | 0.1000 | 0.1000 | 0.1000 |
| Granular | | | | | |
| Pepper, Black, 30-60 mesh | 0.1000 | 0.1000 | 0.1000 | 0.1000 | 0.1000 |
| Parsley, Dehydrated, Granular −10 +30 | 0.0250 | 0.0250 | 0.0250 | 0.0250 | 0.0250 |
| Preservative, EDTA, Calcium Disodium | 0.0070 | 0.0070 | 0.0070 | 0.0070 | 0.0070 |
| TOTAL | 100.0000 | 100.0000 | 100.0000 | 100.0000 | 100.0000 |

TABLE 7b
Ranch Dressing Production Process

| | |
|---|---|
| 1. | Check that the Mixer is in good working condition, free and |
| | clear of any dirt or dust, sealed tight. |
| 2. | Set colloid mill at 0.45″ |
| 3. | Set mix tank speed at 45 hz. |
| 4. | Meter water into the mix tank. |
| 5. | Add in preservatives (Benzoate, Sorbate, EDTA) into the mix tank. |
| 6. | Make gum slurry (Xanthan gum + 700 g soybean oil) |
| 7. | Add slurry to dixie tank, allow to mix for 3 minutes |
| 8. | Increast tank speed to 35 hz. |
| 9. | Add remaining dry ingredients slowly to the mix tank. |
| 10. | Add Egg Yolk and Cultured Milk Powder |
| 11. | Increase tank speed to 45 hz. |
| 12. | Slowly add the remaining soybean oil, and if appropriate, |
| | the Omega 3 oil. |
| 13. | Add slowly, the vinegar and phosphoric acid. |
| 14. | Alll to mix until all ingredients are incorporated and mixed (approx |
| | 30 sec) |
| 15. | Open mix tank valve, and set pump speed to 30 hz. |

TABLE 7c
SHELF LIFE PRODUCTION
ANALTYICAL/MICRO RESULTS
RANCH DRESSING

| | Control | SDA | Fish Oil | Algal Oil | Flax Oil |
|---|---|---|---|---|---|
| | 50-RA-325- | 50-RA-326- | 50-RA-328- | 50-RA-330- | 50-RA-327- |
| | 000 | 000 | 000 | 000 | 000 |
| pH | 3.80 | 3.79 | 3.79 | 3.79 | 3.80 |
| Total Acidity | 0.82 | 0.83 | 0.82 | 0.84 | 0.84 |
| Total Solids | 2.17 | 2.15 | 2.15 | 2.14 | 2.17 |
| Bostwick (viscosity) | 8.3 CM | 8.5 cm | 8.8 cm | 8.5 cm | 8.8 cm |
| Total Plate Count | 30 | 50 | 110 | 30 | 20 |
| Lactics | <10 | <10 | <10 | <10 | <10 |
| Yeast | <10 | <10 | <10 | <10 | <10 |
| Mold | <10 | <10 | <10 | <10 | <10 |

The general approach to the shelf life testing is for 5 trained attribute panelists to taste the dressings and come to consensus regarding the attributes and intensity (on a 15 pt scale—0 being absent, 15 being extreme) for each dressing. The lists of attributes identified by the panelists are in the attached documents. Additional attributes would be identified as warranted.

For the current example the tables above provide significant data on flavor and consistency. In the case of Ranch Dressing, because of its more sensitive flavor, the differences between the dressings made with SDA and the competitive counterparts are more obvious. The tables above represent the data developed for a preferred embodiment of the current invention. Please also see FIGS. 3a-3h for graphical representation of the data with Ranch Dressing. According to the data provided herein the samples containing SDA are significantly less off-flavored than those containing the fish and algal oils. Due to pungent flavors and extremely unpleasant odor the fish and algal derived oils were simply removed from the 3 months accelerated evaluation period whereas SDA was not. Demonstrating improved stability, reduced degradation and consequent enhanced shelf-life.

### Example 3

### Mayonnaise

According to the current invention, a mayonnaise was prepared and tested with the omega-3 containing oil of the invention, the data provided applies for all mayonnaise and spoonable salad dressing variants, produced in a variety of ways (colloid mill, frying mill, etc).

TABLE 8a
SDA - Mayonnaise, Formulation
MAYONNAISE SHELF LIFE
ATTRIBUTES
n = 5

| | Soybean Oil | | | | | SDA | | | | |
|---|---|---|---|---|---|---|---|---|---|---|
| | (reference) | | | | | Oil | | | | |
| | | 95° F. | 95° F. | 73° F. | 73° F. | | 95° F. | 95° F. | 73° F. | 73° F. |
| | Ini | 1 mo | 2 mo | 2 mo | 4 mo | Ini | 1 mo | 2 mo | 2 mo | 4 mo |
| APPEARANCE | | | | | | | | | | |
| Color | 4 | 4.5 | 5 | 4 | 4 | 4 | 4.5 | 5 | 4 | 4 |
| AROMA | | | | | | | | | | |
| Total Aroma | 6 | 6.5 | 7 | 6 | 6 | 6 | 7 | 8.5 | 6.5 | 6.5 |
| Eggy Aroma | 3.5 | 3.5 | 3 | 3.5 | 3 | 3.5 | 3.5 | 2 | 3.5 | 2.5 |
| Vinegar Aroma | 3 | 3.5 | 2.5 | 3 | 3 | 3 | 2.5 | 2.5 | 3 | 2.5 |
| Pungent | 4 | 4.5 | 4 | 4 | 4.5 | 3.5 | 4 | 4.5 | 3.5 | 4.5 |
| Total Oil | 1.5 | 2.5 | 3.5 | 2 | 2.5 | 1.5 | 2.5 | 5 | 2 | 3.5 |
| Total Off | 0.5 | 2 | 3.5 | 1.5 | 2.5 | 0.5 | 3 | 6.5 | 2 | 4.5 |
| Oxidized Oil | 0.5 | 2 | 3.5 | 1.5 | 2 | 0.5 | 2.5 | 5 | 2 | 3.5 |
| FLAVOR | | | | | | | | | | |
| Total Flavor | 6.5 | 7 | 7 | 7 | 7 | 6.5 | 8.5 | 9 | 7 | 8 |
| Eggy Flavor | 4 | 4 | 3 | 4 | 3.5 | 4 | 4.5 | 2.5 | 4 | 3 |
| Vinegar Flavor | 2.5 | 3 | 2.5 | 3 | 2.5 | 2.5 | 2.5 | 2.5 | 2.5 | 2.5 |
| Sweet | 3.5 | 3.5 | 3.5 | 3.5 | 3 | 3.5 | 5 | 3.5 | 3 | 3 |
| Sour | 2.5 | 2.5 | 3 | 3 | 3 | 2.5 | 3.5 | 3 | 2.5 | 3 |
| Salty | 3 | 3 | 3 | 3.5 | 3.5 | 3.5 | 3.5 | 3 | 3.5 | 4 |
| Total Oil | 3 | 3.5 | 4 | 3.5 | 3.5 | 3.5 | 4 | 5.5 | 3.5 | 4.5 |
| Total Off | 1.5 | 3 | 4.5 | 2 | 3.5 | 1 | 5 | 6.5 | 2.5 | 5.5 |
| Oxidized Oil | 1.5 | 2.5 | 4 | 2 | 3 | 0.5 | 4 | 5.5 | 2 | 4.5 |
| TEXTURE | | | | | | | | | | |
| Viscosity by | 8.5 | 8.5 | 8.5 | 8.5 | 8.5 | 8.5 | 8.5 | 8.5 | 8.5 | 9 |
| Mouth | | | | | | | | | | |
| Oily Mouthfeel | 8.5 | 8.5 | 8.5 | 8.5 | 8.5 | 8.5 | 8.5 | 8.5 | 8.5 | 9 |
| (after 5 | | | | | | | | | | |
| seconds) | | | | | | | | | | |

| | | | | | | |
|---|---|---|---|---|---|---|
| Comments: | old oil, | painty, | slightly | reheated oil, slightly beany | slight | Slight |
| | beany, | cardboard | oxidized, | | sulfur, | sulfur, |
| | slightly | | cardboard | | oxidized | pondy, |
| | waxy | | | | oil, | slightly |
| | | | | | slightly | melted |
| | | | | | beany | plastic |

TABLE 8b
Composition of the Invention - Comparison with
Fish Oil-based Mayonnaise
n = 5

| | Fish Oil | | | | |
|---|---|---|---|---|---|
| | | 95° F. | 95° F. | 73° F. | 73° F. |
| | Ini | 1 mo | 2 mo | 2 mo | 4 mo |
| APPEARANCE | | | | | |
| Color | 4 | 4.5 | 5 | 4 | 4 |
| AROMA | | | | | |
| Total Aroma | 6 | 6.5 | 7.5 | 6.5 | 6.5 |
| Eggy Aroma | 3.5 | 3.5 | 3 | 3.5 | 3 |
| Vinegar Aroma | 3 | 3 | 3 | 3 | 3 |
| Pungent | 3.5 | 4 | 4.5 | 4 | 4.5 |
| Total Oil | 1.5 | 2 | 4 | 2.5 | 3 |
| Total Off | 0.5 | 2 | 4.5 | 2 | 3.5 |
| Oxidized Oil | 0.5 | 1.5 | 4 | 2 | 3 |
| FLAVOR | | | | | |
| Total Flavor | 6.5 | 7.5 | 8 | 7.5 | 8 |
| Eggy Flavor | 4 | 4 | 2.5 | 4 | 3 |
| Vinegar Flavor | 2.5 | 2.5 | 2.5 | 2.5 | 2.5 |
| Sweet | 3.5 | 3.5 | 3.5 | 3.5 | 3 |
| Sour | 2.5 | 3.5 | 3.5 | 3 | 3 |
| Salty | 3 | 3.5 | 4 | 3.5 | 4 |
| Total Oil | 3 | 3.5 | 5 | 4 | 5 |
| Total Off | 1 | 3 | 6 | 3.5 | 5.5 |
| Oxidized Oil | 0.5 | 2.5 | 5 | 3.5 | 5 |
| TEXTURE | | | | | |
| Viscosity by | 8.5 | 9 | 8.5 | 8.5 | 9 |
| Mouth | | | | | |
| Oily Mouthfeel | 8.5 | 9 | 8.5 | 8.5 | 9.5 |
| (after 5 seconds) | | | | | |
| Comments: | | fishy, | strong fishy | oxidized oil, | fishy |
| | | musty, | | painty, old | |
| | | painty | | mayo, fish | |

TABLE 8c
Composition of the Invention - Comparison with
Algal Oil-based Mayonnaise
n = 5

| | Algal Oil | | | | |
|---|---|---|---|---|---|
| | | 95° F. | 95° F. | 73° F. | 73° F. |
| | Ini | 1 mo | 2 mo | 2 mo | 4 mo |
| APPEARANCE | | | | | |
| Color | 5.5 | 7 | 6.5 | 6 | 5.5 |
| AROMA | | | | | |
| Total Aroma | 6 | 8 | 9 | 7 | 8 |
| Eggy Aroma | 4 | 2.5 | 2 | 3 | 2 |
| Vinegar Aroma | 3 | 3 | 2.5 | 3 | 2 |
| Pungent | 3.5 | 4.5 | 5 | 4 | 5.5 |
| Total Oil | 1.5 | 4 | 6 | 2.5 | 5 |
| Total Off | 0.5 | 4.5 | 6.5 | 2 | 5.5 |
| Oxidized Oil | 0.5 | 4.5 | 6 | 2 | 5 |
| FLAVOR | | | | | |
| Total Flavor | 6.5 | 9 | 9.5 | 8 | 9 |
| Eggy Flavor | 5 | 2.5 | 2 | 3 | 2 |
| Vinegar Flavor | 2.5 | 2.5 | 2 | 2.5 | 1.5 |
| Sweet | 4 | 2.5 | 3.5 | 3 | 3 |
| Sour | 2.5 | 3.5 | 3.5 | 3 | 3.5 |
| Salty | 3.5 | 3.5 | 3.5 | 3.5 | 4 |
| Total Oil | 3 | 6 | 7 | 5 | 6.5 |
| Total Off | 1.5 | 6.5 | 7.5 | 4.5 | 7.5 |
| Oxidized Oil | 1 | 6 | 7 | 4.5 | 6.5 |
| TEXTURE | | | | | |
| Viscosity by | 8.5 | 8.5 | 8.5 | 8.5 | 9 |
| Mouth | | | | | |
| Oily Mouthfeel | 8.5 | 9 | 8.5 | 8.5 | 8.5 |
| (after 5 seconds) | | | | | |
| Comments: | | fishy, | Strong fishy | Oxidized oil, | Fishy, |
| | | pondy | | painty old | pondy, |
| | | | | mayo, fishy | beany, |
| | | | | | cardboard |

8d Composition of the Invention - Comparison with
Flax Oil-based Mayonnaise
n = 5

| | Flax Oil | | | | |
|---|---|---|---|---|---|
| | | 95° F. | 95° F. | 73° F. | 73° F. |
| | Initial | 1 mo | 2 mo | 2 mo | 4 mo |
| APPEARANCE | | | | | |
| Color | 4.5 | 5.5 | 5.5 | 5 | 5 |
| AROMA | | | | | |
| Total Aroma | 6 | 6.5 | 7.5 | 6.5 | 6.5 |
| Eggy Aroma | 3.5 | 4 | 2 | 3.5 | 2.5 |
| Vinegar Aroma | 3 | 3 | 2.5 | 3.5 | 2.5 |
| Pungent | 3.5 | 4 | 5 | 4.5 | 4 |
| Total Oil | 1.5 | 2.5 | 4.5 | 2 | 3 |
| Total Off | 1.5 | 2 | 5 | 1.5 | 3.5 |
| Oxidized Oil | 1 | 2 | 4.5 | 1.5 | 3 |
| FLAVOR | | | | | |
| Total Flavor | 7 | 7 | 8 | 7.5 | 7.5 |
| Eggy Flavor | 3.5 | 4 | 2.5 | 3.5 | 3 |
| Vinegar Flavor | 2.5 | 2.5 | 2 | 3 | 2.5 |
| Sweet | 3 | 3.5 | 3.5 | 3.5 | 3.5 |
| Sour | 2.5 | 3 | 3 | 3 | 3 |
| Salty | 3.5 | 3.5 | 3.5 | 3.5 | 4 |
| Total Oil | 3 | 3.5 | 5 | 4 | 4.5 |
| Total Off | 3.5 | 2.5 | 5.5 | 3 | 4.5 |
| Oxidized Oil | 3 | 2.5 | 5 | 3 | 4.5 |
| TEXTURE | | | | | |
| Viscosity by Mouth | 8.5 | 9 | 8.5 | 8 | 8.5 |
| Oily Mouthfeel | 8.5 | 9 | 8.5 | 8.5 | 8.5 |
| (after 5 seconds) | | | | | |
| Comments: | | Old oil, | Fishy, | Fishy, | Strong |
| | | reheated oil, | cardboard, | pondy | fishy |
| | | beany, waxy | reheated | | |
| | | | oil | | |

TABLE 9a
SDA MAYONNAISE FORMULATIONS AND
PROCESS

| | Variant | | | | |
|---|---|---|---|---|---|
| | Control | SDA | Fish | Algal | Flax |
| | Code Number | | | | |
| | 050-RA787- | 050-RA788- | 050-RA790- | 050-RA791- | 050-RA792- |
| | 000 | 000 | 000 | 000 | 000 |
| Ingredient | % | % | % | % | % |
| Soy Salad Oil | 80.0000 | 55.6500 | 76.9000 | 77.3500 | 76.7300 |
| Omega 3 Oil | | 24.3500 | 3.1000 | 2.6500 | 3.2700 |
| Water | 7.7930 | 7.7930 | 7.7930 | 7.7930 | 7.7930 |
| Egg Yolk (10% salted) | 6.0000 | 6.0000 | 6.0000 | 6.0000 | 6.0000 |
| White Distilled Vinegar, 120 gr | 3.0000 | 3.0000 | 3.0000 | 3.0000 | 3.0000 |
| High Fructose Corn Syrup - 42 solids | 2.0000 | 2.0000 | 2.0000 | 2.0000 | 2.0000 |
| Salt | 0.8000 | 0.8000 | 0.8000 | 0.8000 | 0.8000 |
| Mustard Flour | 0.4000 | 0.4000 | 0.4000 | 0.4000 | 0.4000 |
| EDTA, Calcium Disodium | 0.0070 | 0.0070 | 0.0070 | 0.0070 | 0.0070 |
| Total | 100.0000 | 100.0000 | 100.0000 | 100.0000 | 100.0000 |

TABLE 9b
Mayonnaise Process - Pilot Plant

| | |
|---|---|
| 2. | Set the colloid mill at 30. |
| 3. | Add the water first, then mix in the EDTA. |
| 4. | Add the egg yolk, mix for 3 min. |
| 5. | Pre-mix the mustard flour, sugar, and salt. Add the premix slowly |
| | until dissolved and evenly dispersed. |
| 6. | Add in the oils mix for 3 minutes, set Dixie mix tank speed at 35 hz. |
| 7. | Slowly add in the vinegar |
| 8. | Mix until all ingredients are dispersed. Shut off Dixie Mixer |
| | agitation, allow air to escape. |
| 9. | Start up the Collid Mill. Open mix tank, valve, set pump speed to |
| | 30 hz. |
| 10. | Pack into individual packages. |

According to the current invention. The general approach to the shelf life testing is for 5 trained attribute panelists to taste the dressings and come to consensus regarding the attributes and intensity (on a 15 pt scale—0 being absent, 15 being extreme) for each dressing. The lists of attributes identified by the panelists are in the attached documents. Additional attributes would be identified as warranted.

TABLE 9c

| | VALUE | SCALE REFERENCE |
|---|---|---|
| APPEARANCE | | |
| Color | 0.0 | White (paper) |
| | 7.5 | Manila Folder |
| AROMA\FLAVOR | | |
| Eggy | 8.0/6.0 | Chopped Hard Boiled Eggs |
| Vinegar Aroma | 6.5 | 100% Heinz Distilled Vinegar solution |
| Vinegar Flavor | 4.0 | 2% Heinz Distilled Vinegar solution |
| Total Off | 3.5 | Edamame, raw soybeans |
| Oxidized Dairy/Oil | 4.0 | Canola Oil |
| | | (opened September 2005) |
| (aroma and flavor) | 5.0 | Wesson Vegetable Oil |
| | | (opened Nov. 22, 2004) |
| | 8.0 | Kraft Parmesan Cheese |
| | | (2001 expiration date) |
| Sweet | 2.0 | 2.0% Sucrose in Water |
| | 5.0 | 5.0% Sucrose in Water |
| Sour | 2.0 | 0.025% Citric Acid in Water |
| | 5.0 | 0.04% Citric Acid in Water |
| Salty | 2.0 | 0.2% Sodium Chloride in Water |
| | 5.0 | 0.5% Sodium Chloride in Water |
| MOUTHFEEL | | |
| FACTORS | | |
| Pungent (aroma) | 8.0 | 100% Heinz Distilled Vinegar solution |
| TEXTURE | | |
| Viscosity by Mouth | 8.0 | 50:50 mix of Lucerne Heavy Cream |
| | | and Kraft Mayonnaise |
| | 11.0 | Kraft Mayonnaise |
| Oily Mouthfeel | 8.0 | Kraft Mayonnaise |

According to the current invention the following data was developed after initial evaluations. Similar to the Salad Dressings example, the initial flavor of SDA containing mayonnaise was similar to the control. The flax sample was most different from the others compared

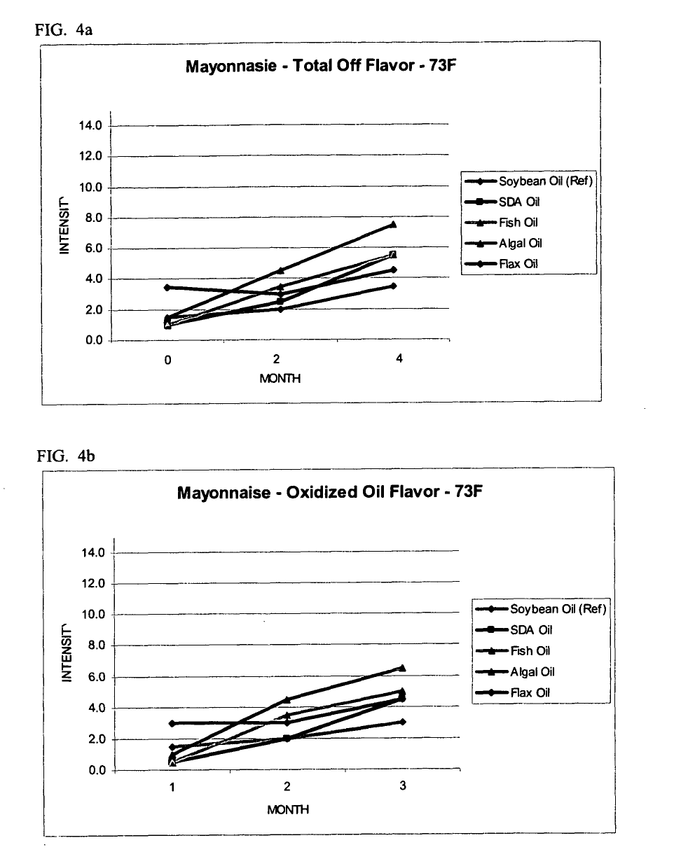

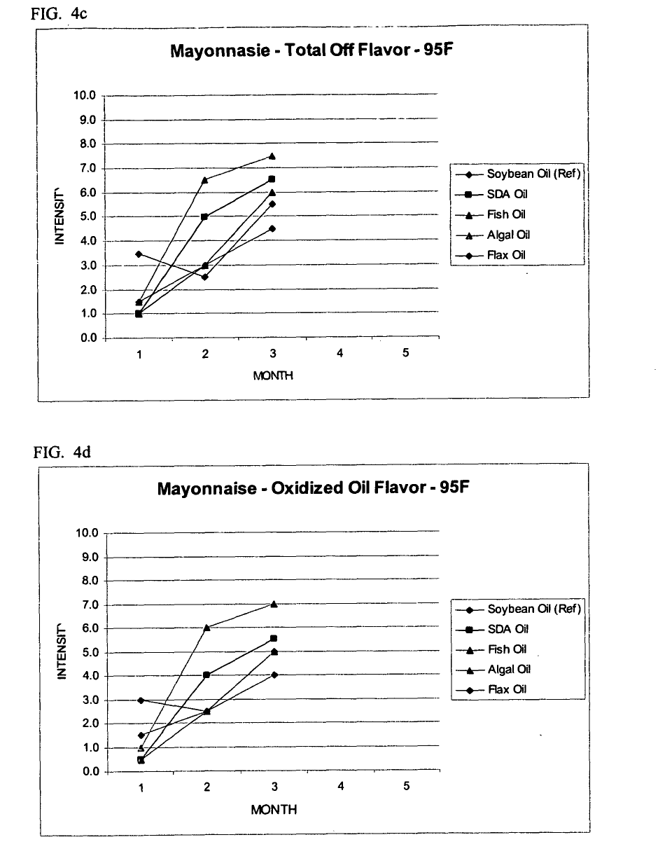

According to the methods of the current invention, the shelf-life studies two month studies at both room temperature and accelerated storage conditions were completed. All samples in the accelerated temperature study had noticeable off flavor with the algal oil sample containing the highest off notes. SDA performed better than the other omega-3 containing oil sources. For the room temperature study, Algal oil exhibited much higher levels of off flavors than the SDA oil of the invention. See the above data in tables 12-14 and FIGS. 4a-4e.

### Example 4

### Soy Milk

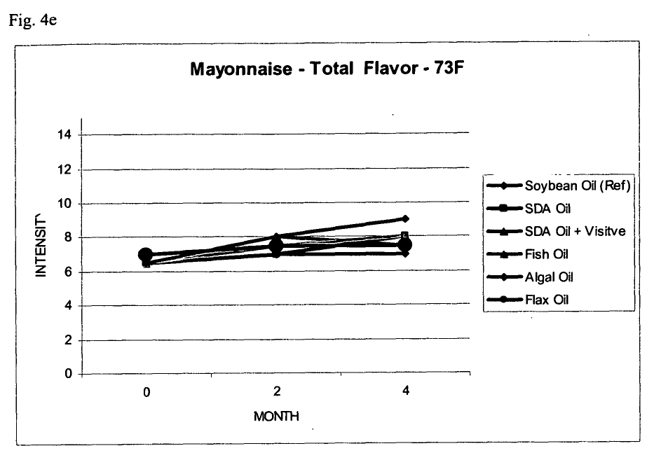

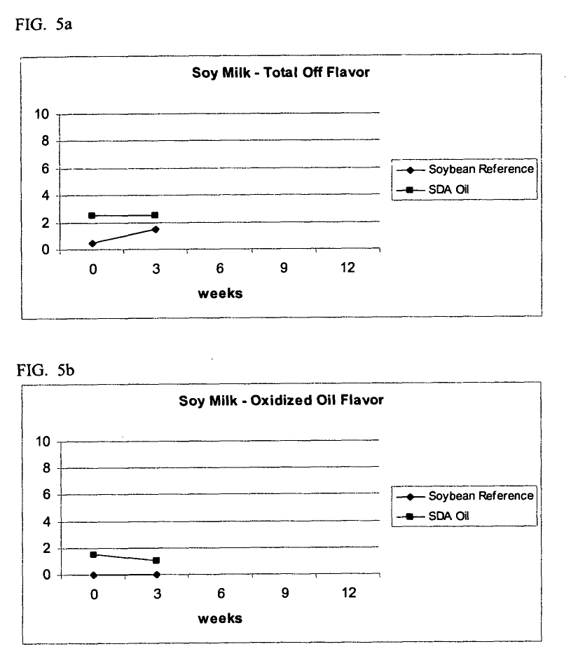

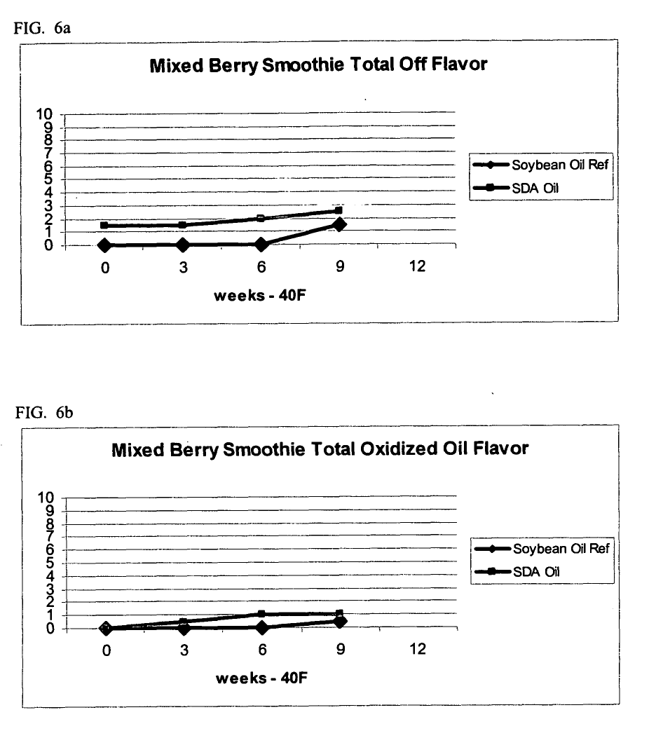

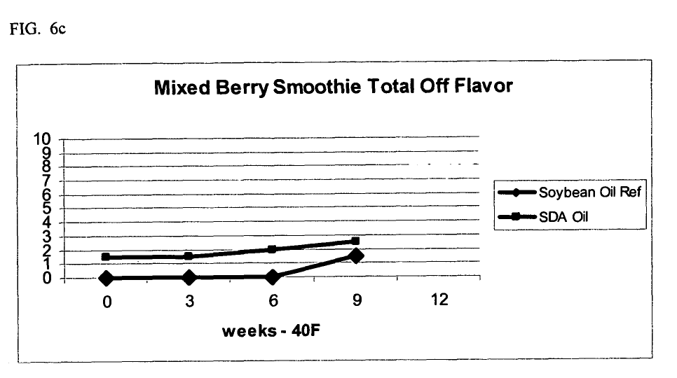

According to the current invention, Soymilk can be prepared in two different ways. In the first, SDA enriched soybeans are de-hulled, flaked and then made into full fatted soy flour. The soymilk is formulated by first dissolving the soy flour into water, mixing, and processing to inactivate the enzymes. The soy base is filtered to remove additional solids and degassed. The remaining ingredients are added, mixed, the product is then homogenized in a two stage homogenizer, then processed through a Ultra High Temperature (UHT) thermal processing unit. The resulting product is packed and refrigerated with a typical shelf life of 12 weeks. Following is a formulation as provided in Table 10, see also FIG. 6 for a process flow diagram.

TABLE 10

| Vanilla Soymilk | % |
|---|---|
| Water | 88.122 |
| SDA Enriched Soy Flour | 6.786 |
| Full Fat Soymilk. | 0.600 |
| Sucrose | 3.400 |
| Carageenen | 0.022 |
| Cellulose Gum | 0.350 |
| Salt | 0.040 |
| Calcium Carbonate | 0.350 |
| Natural and Artificial Flavors | 0.330 |
| TOTAL | 100.000 |

The example used can also be applied to different types of homogenization and thermal processing units (direct steam, indirect steam, etc.). Different soymilk flavors, including plain, chocolate, apple, orange, beny, etc. can be prepared in the same manner.

The resulting product was found to have acceptable flavor and mouth “feel” properties in comparison to soymilk made from flour processed the same way but without the SDA enhancement of the current invention. According to the data developed in pursuit of the current invention after 9 months shelf life, only slight differences in taste exist between the embodiments of the current invention enhanced with a transgenic SDA composition versus a control composition with non-transgenic soybean oil containing no Omega-3 fatty acids. This was done for both the soymilk and fruit smoothies. Note these are kept refrigerated and only have a 3 month shelf life in most commercial settings.

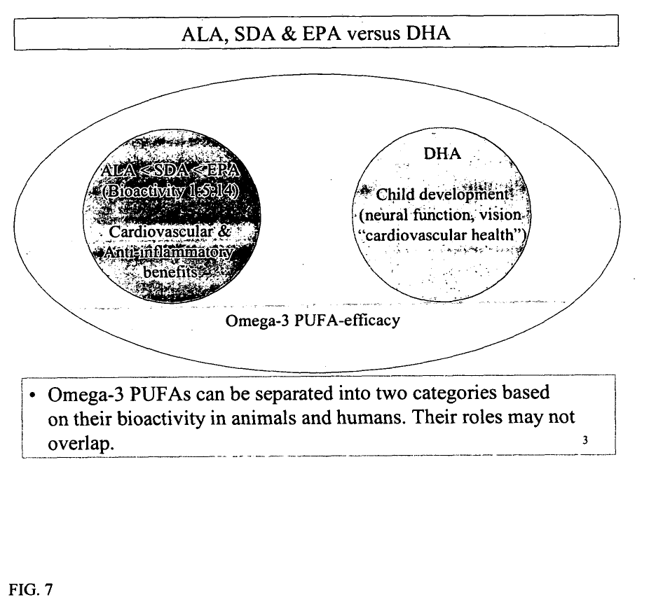

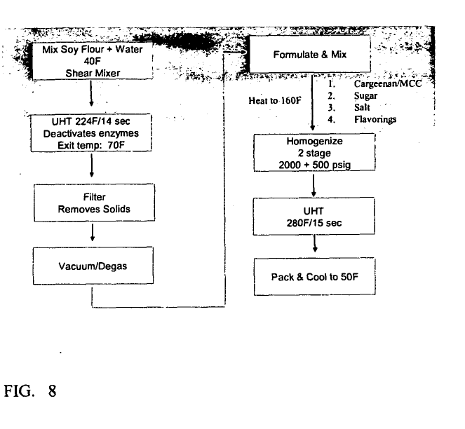

The second approach to this example is to use isolated soy protein, and to add SDA enriched soy oil to achieve a new product composition. Following is a formulation as provided in Table 11 with a corresponding flow diagram in FIG. 7.

TABLE 11

| Vanilla Soymilk | % |
|---|---|
| Water | 88.058 |
| Sucrose | 3.500 |
| Isolated Soy Protein | 2.700 |
| Maltodextrin | 3.500 |
| 11% SDA Soybean Oil | 1.500 |
| Caraeenan | 0.022 |
| Cellulose gum | 0.350 |
| Salt | 0.040 |
| Natural & Artificial Flavors | 0.330 |
| TOTAL | 100.000 |

According to the current invention the example provided above used can also be applied to different types of homogenization and thermal processing units (direct steam, indirect steam, etc.). Different soymilk flavors, including plain, chocolate, apple, orange, berry, etc. can be prepared in the same manner. The resulting product was found to have acceptable flavor and mouthfeel properties in comparison to soymilk made with refined, bleached and deodorized soybean oil.

TABLE 12
SOYMILK ATTRIBUTE REMARKS AND EVALUATION
9 WEEK PROFILES
(n = 5 panelists)

| | Soybean Oil | | | | SDA Oil | | | |
|---|---|---|---|---|---|---|---|---|
| | (reference) | 40° F. | 40° F. | 40° F. | | 40° F. | 40° F. | 40° F. |
| | Initial | 3 wks | 6 wks | 9 wks | Initial | 3 wks | 6 wks | 9 wks |
| APPEARANCE | | | | | | | | |
| Color | 4 | 4 | 4 | 4 | 4 | 4 | 4 | 4 |
| AROMA | | | | | | | | |
| Total Aroma | 8.5 | 8.5 | 8.5 | 8.5 | 7.5 | 6.5 | 6.5 | 7.5 |
| Sweet | 7.5 | 7.5 | 7.5 | 7.5 | 6 | 6 | 6 | 6 |
| Aromatic/Vanilla | | | | | | | | |
| Cereal/Soy/Grain | 4.5 | 4.5 | 4.5 | 4.5 | 4.5 | 4.5 | 4.5 | 4.5 |
| Total Off | 0 | 0 | 0 | 0 | 2.5 | 2 | 2 | 1 |
| Oxidized Oil | 0 | 0 | 0 | 0 | 2 | 1 | 1 | 1 |
| FLAVOR | | | | | | | | |
| Total Flavor | 7.5 | 7.5 | 6.5 | 7 | 8.5 | 6 | 6 | 6.5 |
| Sweet | 5.5 | 5.5 | 5 | 4.5 | 5.5 | 4.5 | 4 | 4 |
| Aromatic/Vanilla | | | | | | | | |
| Cereal/Soy/Grain | 6.5 | 6.5 | 6 | 6.5 | 7 | 5.5 | 5.5 | 5.5 |
| Sweet | 5 | 5 | 4.5 | 4.5 | 5 | 4.5 | 4 | 4 |
| Bitter | 0.5 | 1.5 | 1.5 | 1.5 | 1.5 | 3 | 2 | 2 |
| Total Off | 0.5 | 1.5 | 1.5 | 1.5 | 2.5 | 2.5 | 2.5 | 2 |
| Oxidized Oil | 0 | 0 | 1 | 1 | 1.5 | 1 | 1.5 | 1.5 |
| TEXTURE | | | | | | | | |
| Viscosity by Mouth | 2 | 2 | 2 | 2 | 2 | 2 | 2 | 2 |
| Chalky/Gritty | 3 | 3 | 2.5 | 3.5 | 3 | 3 | 2.5 | 3 |
| Astringent | 4 | 4 | 4 | 4 | 4 | 4 | 4 | 4 |
| Comments: | slight | cardboard | slight | slight | slightly | cardboard, | beany, | beany, |
| | green, | packaging | soft | cardboard | chemical, | playdough, | cardboard | cardboard |
| | slight | | plastic, | | slightly | slight | | |
| | beany | | slight | | oxidized | chemical/solvent | | |
| | | | waxy | | oil, | | | |
| | | | | | slightly | | | |
| | | | | | pondy | | | |

Scale range = 0 to 15
Note:
color indicates variance from Soybean reference; yellow = +/−1.0, orange = +/−1.5 to 2.0, red = /<2.5

### Example 5

### Fruit Smoothies

According to a preferred embodiment of the current invention, fruit smoothies, developed from soymilk. Other sources of SDA oil could be used for the development of fruit smoothies as well, in alternative embodiments. Also according to the current invention the processes developed for the production of the fruit smoothies takes into account the unique properties of the SDA oil for enhancing health and nutrition. Two smoothie type products have been developed, and both products have been determine to have extended shelf life properties. During a process that involves the utilization of ultra high pasteurization, stored refrigerated, with a 12 week shelf life typical of other refrigerated drinks. Although a mixed berry prototype is described herein, other flavors can be developed including strawberry, grape, cranberry, orange, lemon, apple, pineapple, mango, strawberry-banana and any other fruit flavor combination.

In the first approach, soymilk is prepared as described in the first part of Example 4, utilizing SDA enriched soy flour. Additional ingredients including stabilizers, flavorings and fruit are added prior to homogenization. The following is a formulation used for the product:

TABLE 13
MIXED BERRY FRUIT SMOOTHIE - SOY BASED

| | % |
|---|---|
| Water | 77.774 |
| SDA Enriched Soy Flour | 6.773 |
| Pectin | 0.300 |
| Cellulose gel/pectin mix | 0.400 |
| Sucrose | 9.300 |
| Citric Acid, anhydrous | 0.450 |
| Potassium Citrate, granular | 0.060 |
| Soy lecithin | 0.060 |
| Salt | 0.070 |
| Frozen Strawbery Puree | 4.000 |
| Frozen Blackberry Puree | 0.500 |
| Red Grape Juice Concentrate | 0.123 |
| Natural Flavor | 0.020 |
| Natural Flavor | 0.060 |
| Natural Berry Flavor | 0.050 |
| Natural and Artificial Mixed Berry Flavor | 0.040 |
| Natural and Artificial Blueberry Flavor | 0.020 |
| Total | 100.000 |

The soybase portion was prepared according to the process described in Example 4. The processing for the remainder of the product is described below:

TABLE 14
Preparation Procedures:

| | |
|---|---|
| 1. | Pre-weigh all dry ingredients |
| 2. | Stabilizer portion: Add prescribed water for stabilizer portion into |
| | mixing vessel and begin agitation. |
| 3. | Heat water to 110 to 120° F. |
| 4. | Mix the pectin and Avicel with a portion of the dry sugar and add |
| | slowly to the water with high shear mixing. Allow 5 minutes for |
| | hydration. |
| 5. | Add the citric acid. |
| 6. | Soy milk portion: |
| 7. | Add the potassium citrate, soy lecithin and salt. |
| 8. | Combine the stablilzer portion and soymilk portion into larger, steam |
| | jacketed mixing vessel. |
| 9. | Add the purees, color, and flavorings and mix until uniform. |
| 10. | Check pH. Expected pH 4.2 ± 0.2. |
| 11. | Heat to 160° F. and homogenize d/s 2500 + 500 psi. (3000 psi total) |
| 12. | UHT process in the Microthermics unit. Target process is 224° F. |
| | for 19 seconds. |
| 13. | Cool in Microthermics cooling sections and fill directly into |
| | containers. |
| 14. | Apply closure and place bottles into chilled water bath. |
| | Cool to ≦50° F. |
| 15. | Take count of bottles, apply labels, and refrigerate |
| | (PD Warehouse walk-in refrigerator). |

A second approach developed by the current invention is where an SDA enriched oil is added to a formulation containing Isolated Soy Protein. In this embodiment, a mixed berry product was developed, but can be extended to additional flavors as described above. Following is the basic formulation used in an embodiment of the current invention:

TABLE 15
MIXED BERRY FRUIT SMOOTHIE - SOY BASED

| | % |
|---|---|
| Water | 81.077 |
| Pectin | 0.300 |
| Cellulose gel/pectin mix | 0.400 |
| Sucrose | 8.700 |
| Citric Acid, anhydrous | 0.310 |
| 11% SDA Soybean Oil | 1.500 |
| Isolated Soy Protein | 2.700 |
| Potassium Citrate, granular | 0.060 |
| Soy lecithin | 0.080 |
| Salt | 0.060 |
| Frozen Strawbery Puree | 4.000 |
| Frozen Blackberry Puree | 0.500 |
| Red Grape Juice Concentrate | 0.123 |
| Natural Flavor | 0.020 |
| Natural Flavor | 0.060 |
| Natural Berry Flavor | 0.050 |
| Natural and Artificial Mixed Berry Flavor | 0.040 |
| Natural and Artificial Blueberry Flavor | 0.020 |
| Total | 100.000 |

The product was developed according to the methods of the invention and has the following formulation:

TABLE 16
Preparation Procedures:

| | |
|---|---|
| 1. | Pre-weigh all dry ingredients |
| 2. | Stabilizer portion: Add prescribed water for stabilizer portion into |
| | mixing vessel and begin agitation. |
| 3. | Heat water to 110 to 120° F. |
| 4. | Mix the pectin and Avicel with a portion of the dry sugar and add |
| | slowly to the water with high shear mixing. Allow 5 minutes for |
| | hydration. |
| 5. | Add the citric acid. |
| 6. | Soy milk portion: Add the prescribed water for the soymilk portion |
| | into a separate mixing vessel and begin agitation. |
| 7. | Heat the water to 100 to 110° F. |
| 8. | Add the soy protein isolate. Mix well to disperse. |
| 9. | Add the potassium citrate, soy lecithin, salt and oil. |
| 10. | Combine the stablilzer portion and soymilk portion into larger, |
| | steam jacketed mixing vessel. |
| 11. | Add the frozen strawberry puree, color, and flavorings and mix |
| | until uniform. |
| 10. | Check pH. Expected pH 4.2 ± 0.2. |

The resulting products from both approaches in this example were typical of a fruit flavored smoothie embodiment of the invention with a refrigerated shelf life of 12 months as developed for the current invention.

The data and techniques above demonstrate the production of a mixed berry smoothie from soymilk according to the methods of the invention. According to an embodiment of the invention the SDA oil of the invention provides substantial differences relative to other omega-3 containing samples. The data is presented in Table 17-21 and graphs demonstrating the results are in FIGS. 6a-6b.

TABLE 17
MIXED BERRY SMOOTHIE - ATTRIBUTE RESULTS
9 WEEK PROFILES
(n = 5 panelists)

| | Soybean Oil | | | | SDA Oil | | | |
|---|---|---|---|---|---|---|---|---|
| | (reference) | 40° F. | 40° F. | 40° F. | | 40° F. | 40° F. | 40° F. |
| | Initial | 3 wks | 6 wks | 9 wks | Ini | 3 wks | 6 wks | 9 wks |
| APPEARANCE | | | | | | | | |
| Color | 4 | 4 | 4 | 4 | 4 | 4 | 4 | 4 |
| AROMA | | | | | | | | |
| Total Aroma | 7 | 7 | 7.5 | 7 | 6.5 | 6.5 | 6.5 | 6.5 |
| Sweet Aromatic | 5.5 | 5.5 | 6 | 5.5 | 4.5 | 5 | 4.5 | 4.5 |
| Berry* | 6.5 | 6.5 | 7 | 6.5 | 5.5 | 5 | 5 | 5.5 |
| Total | 2.5 | 2.5 | 2.5 | 2.5 | 2 | 2 | 2.5 | 2.5 |
| Dairy/Cultured | | | | | | | | |
| Dairy | | | | | | | | |
| Cereal/Soy/Grain | 1.5 | 1.5 | 1.5 | 1.5 | 1 | 2 | 1.5 | 1.5 |
| Total Off | 0 | 0 | 0 | 0 | 0.5 | 1 | 0.5 | 0.5 |
| Oxidized Oil | 0 | 0 | 0 | 0 | 0 | 0.5 | 0 | 0.5 |
| FLAVOR | | | | | | | | |
| Total Flavor | 8.5 | 8.5 | 9 | 8.5 | 8 | 7.5 | 8 | 8 |
| Sweet Aromatic | 6.5 | 6.5 | 6 | 6.5 | 6 | 5.5 | 5 | 5.5 |
| Berry* | 7.5 | 7.5 | 7.5 | 7.5 | 7 | 6.5 | 6.5 | 6.5 |
| Total | 4 | 4 | 4.5 | 4 | 3.5 | 4 | 4 | 4 |
| Dairy/Cultured | | | | | | | | |
| Dairy | | | | | | | | |
| Cereal/Soy/Grain | 2.5 | 2.5 | 2.5 | 2.5 | 2 | 2.5 | 3 | 2 |
| Sweet | 8 | 8 | 7.5 | 8 | 7 | 7 | 6.5 | 7 |
| Sour | 5 | 5 | 5.5 | 5.5 | 6 | 5 | 5.5 | 6 |
| Bitter | 1 | 1 | 1 | 1.5 | 1.5 | 1.5 | 2 | 2 |
| Total Off | 0 | 0 | 0 | 1.5 | 1.5 | 1.5 | 2 | 2.5 |
| Oxidized Oil | 0 | 0 | 0 | 0.5 | 0 | 0.5 | 1 | 1 |
| TEXTURE | | | | | | | | |
| Viscosity by | 4 | 4 | 4.5 | 4.5 | 4 | 4 | 4.5 | 4 |
| Mouth | | | | | | | | |
| Chalky/Gritty | 3.5 | 3.5 | 3.5 | 3.5 | 3.5 | 3.5 | 4 | 4 |
| Astringent | 4 | 4 | 4 | 4 | 4 | 4 | 4.5 | 4 |
| Comments: | | | | slight | slightly | slight | beany, | sllight |
| | | | | beany | oxidized | cooked | green | bean, |
| | | | | | milk | berry, | beany, | oxidized |
| | | | | | | cardboard | oxidized | milk |
| | | | | | | | milk | |

### Example 6

### Margarine Type Spreads

TABLE 18
70% Fat Margarine Type Spread

| | Control | SDA | Fish | Algal | Flax |
|---|---|---|---|---|---|
| Ingredient | % | % | % | % | % |
| Soy Salad Oil | 35.00 | 10.65 | 31.90 | 32.35 | 31.73 |
| Partially Hydrogenated | 35.00 | 35.00 | 35.00 | 35.00 | 35.00 |
| Soy Bean Oil* | | | | | |
| Omega 3 Oil | | 24.35 | 3.10 | 2.65 | 3.27 |
| Water | 27.60 | 27.60 | 27.60 | 27.60 | 27.60 |
| Salt | 2.00 | 2.00 | 2.00 | 2.00 | 2.00 |
| Lecithin, Soy Based** | 0.14 | 0.14 | 0.14 | 0.14 | 0.14 |
| Sodium Benzoate | 0.09 | 0.09 | 0.09 | 0.09 | 0.09 |
| 52% Plastic Mono & | 0.15 | 0.15 | 0.15 | 0.15 | 0.15 |
| Diglyceride*** | | | | | |
| Vitamin A/Beta | 0.01 | 0.01 | 0.01 | 0.01 | 0.01 |
| Carotene Blend**** | | | | | |
| Natural & Artificial | 0.01 | 0.01 | 0.01 | 0.01 | 0.01 |
| Butter Flavor | | | | | |
| Total | 100.00 | 100.00 | 100.00 | 100.00 | 100.00 |

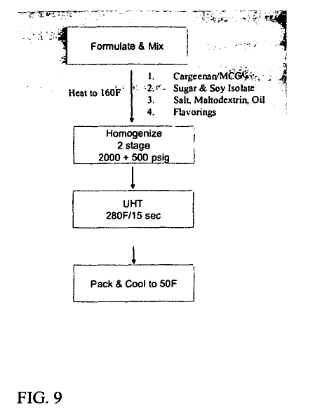

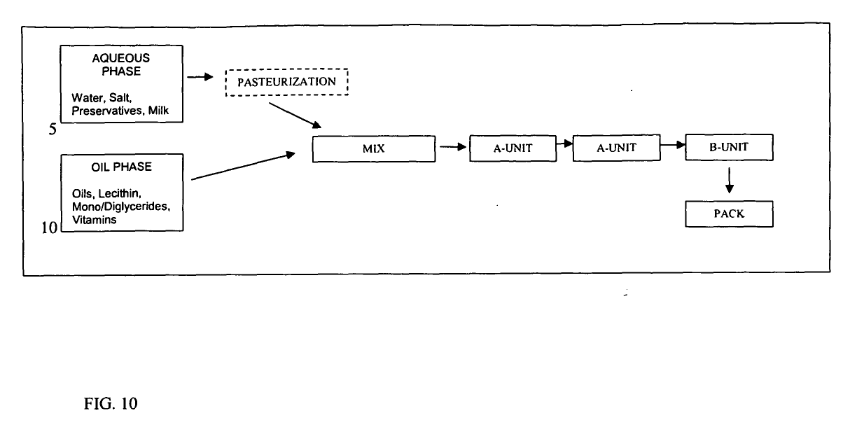

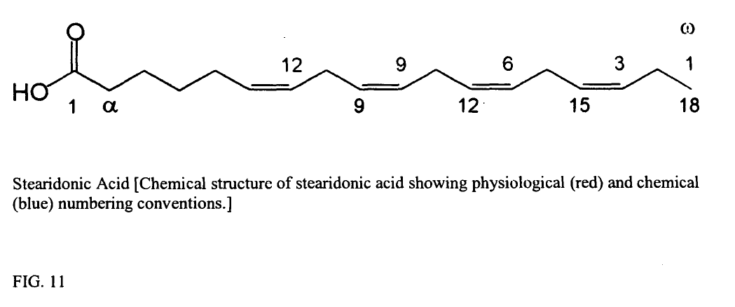

According to a preferred embodiment of the current invention, a typical margarine process, is, the water, salt, sodium benzoate, and butter flavor are mixed as an aqueous phase. Turning to FIG. 9 a milk ingredient, such as whey powder, sodium caseinate or milk powder may be added to the aqueous phase. The oils, lecithin, mono and diglycerides, vitamins, and flavorings are mixed, and combined with the aqueous phase and mixed. The mixed emulsion is passed through a series of scraped surface heat exchangers, pin mixers and resting tubes (A, B and C units respectively) to achieve a desired fill temperature and consistency.

### Example 7

### Cookie Dough

According to the invention the SDA oil of the invention can also be developed into food products including cookies. Below is provided one recipe for such utilization.

TABLE 19

| Ingredient | % |
|---|---|
| Flour | 49.20 |
| Baker's Sugar | 16.00 |
| Hardened soybean oil (Mpt 36-38°) | 17.40 |
| 20% SDA Oil | 7.5 |
| Liquid soya oil | 4.1 |
| Salt | 0.80 |
| Water | 5.00 |
| Total | 100.00 |

### Recombinant Plant Production

One method to recombinantly produce a protein of interest a nucleic acid encoding a transgenic protein can be introduced into a host cell. The recombinant host cells can be used to produce the transgenic protein, including a desirable fatty acid such as SDA that can be secreted or held in the seed, seed pod or other portion of a target plant. A nucleic acid encoding a transgenic protein can be introduced into a host cell, e.g., by homologous recombination. In most cases, a nucleic acid encoding the transgenic protein of interest is incorporated into a recombinant expression vector.

In particular the current invention is also directed to transgenic plants and transformed host cells which comprise, in a 5′ to 3′ orientation, a promoter operably linked to a heterologous structural nucleic acid sequence. Additional nucleic acid sequences may also be introduced into the plant or host cell along with the promoter and structural nucleic acid sequence. These additional sequences may include 3′ transcriptional terminators, 3′ polyadenylation signals, other untranslated nucleic acid sequences, transit or targeting sequences, selectable markers, enhancers, and operators.

Preferred nucleic acid sequences of the present invention, including recombinant. vectors, structural nucleic acid sequences, promoters, and other regulatory elements, are described above. The means for preparing such recombinant vectors are well known in the art. For example, methods for making recombinant vectors particularly suited to plant transformation are described in U.S. Pat. Nos. 4,940,835 and 4,757,011.

Typical vectors useful for expression of nucleic acids in cells and higher plants are well known in the art and include vectors derived from the tumor-inducing (Ti) plasmid of Agrobacterium tumefaciens. Other recombinant vectors useful for plant transformation, have also been described in the literature.

The transformed host cell may generally be any cell which is compatible with the present invention. The transformed host cell may be prokaryotic, more preferably a bacterial cell, even more preferably an Agrobacterium, Bacillus, Escherichia, Pseudomonas cell, and most preferably is an Escherichia coli cell. Alternatively, the transformed host cell is preferably eukaryotic, and more preferably a plant, yeast, or fungal cell. The yeast cell preferably is a Saccharomyces cerevisiae, Schizosaccharomyces pombe, or Pichia pastoris. The plant cell preferably is an alfalfa, apple, banana, barley, bean, broccoli, cabbage, canola, carrot, cassava, celery, citrus, clover, coconut, coffee, corn, cotton, cucumber, garlic, grape, linseed, melon, oat, olive, onion, palm, pea, peanut, pepper, potato, radish, rapeseed (non-canola), rice, rye, sorghum, soybean, spinach, strawberry, sugarbeet, sugarcane, sunflower, tobacco, tomato, or wheat cell. The transformed host cell is more preferably a canola, maize, or soybean cell; and most preferably a soybean cell. The soybean cell is preferably an elite soybean cell line. An “elite line” is any line that has resulted from breeding and selection for superior agronomic performance.

The transgenic plant of the invention is preferably an alfalfa, apple, banana, barley, bean, broccoli, cabbage, canola, carrot, cassava, celery, citrus, clover, coconut, coffee, corn, cotton, cucumber, garlic, grape, linseed, melon, oat, olive, onion, palm, pea, peanut, pepper, potato, radish, rapeseed (non-canola), rice, rye, safflower, sorghum, soybean, spinach, strawberry, sugarbeet, sugarcane, sunflower, tobacco, tomato, or wheat plant. The transformed host plant is most preferably a canola, maize, or soybean cell; and of these most preferably a soybean plant.

### Method for Preparing Transgenic Plants

The invention is further directed to a method for preparing transgenic plants capable of producing a substantial amount of SDA comprising, in a 5′ to 3′ direction, a promoter operably linked to a heterologous structural nucleic acid sequence. The nucleic acid sequence comprising the sequence of SDA when translated and transcribed into amino acid form. Other structural nucleic acid sequences may also be introduced into the plant along with the promoter and structural nucleic acid sequence. These other structural nucleic acid sequences may include 3′ transcriptional terminators, 3′ polyadenylation signals, other untranslated nucleic acid sequences, transit or targeting sequences, selectable markers, enhancers, and operators.

The method generally comprises selecting a suitable plant cell, transforming the plant cell with a recombinant vector, obtaining the transformed host cell, and culturing the transformed host cell under conditions effective to produce a plant.

The transgenic plant of the invention may generally be any type of plant, preferably is one with agronomic, horticultural, ornamental, economic, or commercial value, and more preferably is an alfalfa, apple, banana, barley, bean, broccoli, cabbage, canola, carrot, castorbean, celery, citrus, clover, coconut, coffee, corn, cotton, cucumber, Douglas fir, Eucalyptus, garlic, grape, Loblolly pine, linseed, melon, oat, olive, onion, palm, parsnip, pea, peanut, pepper, poplar, potato, radish, Radiata pine, rapeseed (non-canola), rice, rye, safflower, sorghum, Southern pine, soybean, spinach, strawberry, sugarbeet, sugarcane, sunflower, Sweetgum, tea, tobacco, tomato, turf, or wheat plant. The transformed plant is more preferably a canola, maize, or soybean cell; and most preferably a soybean plant. The soybean plant is preferably an elite soybean plant. An elite plant is any plant from an elite line. Elite lines are described above.

The regeneration, development, and cultivation of plants from transformed plant protoplast or explants is well taught in the art (Gelvin et al., PLANT MOLECULAR BIOLOGY MANUAL, (1990); and, Weissbach and Weissbach, METHODS FOR PLANT MOLECULAR BIOLOGY (1989)). In this method, transformants are generally cultured in the presence of a selective media which selects for the successfully transformed cells and induces the regeneration of the desired plant shoots. These shoots are typically obtained within two to four months.

The shoots are then transferred to an appropriate root-inducing medium containing the selective agent and an antibiotic to prevent bacterial growth. Many of the shoots will develop roots. These are then transplanted to soil or other media to allow the continued development of roots. The method, as outlined, will generally vary depending on the particular plant strain employed.

Preferably, the regenerated transgenic plants are self-pollinated to provide homozygous transgenic plants. Alternatively, pollen obtained from the regenerated transgenic plants may be crossed with non-transgenic plants, preferably inbred lines of economically important species. Conversely, pollen from non-transgenic plants may be used to pollinate the regenerated transgenic plants.

The transgenic plant may pass along the nucleic acid sequence encoding the protein of interest to its progeny. The transgenic plant is preferably homozygous for the nucleic acid encoding the protein of interest protein and transmits that sequence to all its offspring upon as a result of sexual reproduction. Progeny may be grown from seeds produced by the transgenic plant. These additional plants may then be self-pollinated to generate a true breeding line of plants.

The progeny from these plants are evaluated, among other things, for gene expression. The gene expression may be detected by several common methods (e.g., western blotting, immunoprecipitation, and ELISA).

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

One skilled in the art can refer to general reference texts for detailed descriptions of known techniques discussed herein or equivalent techniques. These texts include: Ausubel, et al., CURRENT PROTOCOLS IN MOLECULAR BIOLOGY (eds., John Wiley & Sons, N.Y. (1989)); Birren et al., GENOME ANALYSIS: A LABORATORY MANUAL 1: ANALYZING DNA, (Cold Spring Harbor Press, Cold Spring Harbor, N.Y. (1997)); Clark, PLANT MOLECULAR BIOLOGY: A LABORATORY MANUAL, (Clark, Springer-Verlag, Berlin, (1997)); and, Maliga et al., METHODS IN PLANT MOLECULAR BIOLOGY, (Cold Spring Harbor Press, Cold Spring Harbor, N.Y. (1995)). These texts can, of course, also be referred to in making or using an aspect of the invention. It is understood that any of the agents of the invention can be substantially purified and/or be biologically active and/or recombinant.

### Reduction of Linoleic Acid

It is known that Omega-3 and Omega-6 fatty acids are fatty acids that are required in human nutrition. Omega-6 fatty acids include linoleic acid and its derivatives. These oils are considered essential to human nutrition because these fatty acids must be consumed in the diet because humans cannot manufacture them from other dietary fats or nutrients, and they cannot be stored in the body. Fatty Acids of this sort provide energy and are also components of nerve cells, cellular membranes, and are converted to hormone-like substances known as prostaglandins.

Looking at FIG. 1, linoleic acid is an 18-carbon long polyunsaturated fatty acid containing two double bonds. Its first double bond occurs at the sixth carbon from the omega end, classifying it as an omega-6 oil. As linoleic acid is absorbed and metabolized in the human body, it is converted into a derivative fatty acid, gamma linoleic acid (GLA), which is converted into di-homo-gamma linoleic acid (DGLA) and arachidonic acid (AA). The DGLA and AA are then converted into two types of prostaglandins by adding two carbon molecules and removing hydrogen molecules. There are three families of prostaglandins, PGE1, PGE2, and PGE3. DGLA is converted to PGE1, while AA is converted into PGE2. PGE3 is made by the conversion of omega-3 fatty acids.

In humans the over consumption of omega-6 oils in relation to consumption of omega-3 oils can lead to an overproduction of inflammation-producing prostagladins (PGE2) and a scarcity of anti-inflammatory prostaglandins (PGE1 and PGE2). This in turn can lead to a variety of other health problems. Going further, the daily consumption of omega-6 fatty acids by consumers may be excessive, due to the presence of omega-6 fatty acids in common cooking vegetable oils and processed foods currently on the market. The ratio of omega-6 to omega-3 fatty acid consumption can often reach 20:1 in western diets. To achieve a more desirable ratio, an embodiment of the current invention provides for the increased production of SDA while reducing the production of LA in a transgenic oilseed plant. The resulting oil contains lower levels of LA while providing for the production of significant quantities of SDA and can be used in a variety of roles in the food industry from cooking oil to food ingredient.

### Raisins Tocopherol Levels

Tocopherols are natural antioxidants and essential nutrients in the diet found in plant oils. These antioxidants protect cell membranes and other fat-soluble parts of the body, such as low-density lipoprotein (LDL) cholesterol from damage. It also appears to protect the body against cardiovascular disease and certain forms of cancer and has demonstrated immuno-enhancing effects. According to the current invention enhancements in the presence of tocopherols in the oil of transgenic seed oil plants will be beneficial to consumers of the oil. Relative to the purposes of the current invention enhanced concentrations of tocopherols present in various embodiments of the current will be beneficial as a part of an oil product and may also reduce the oxidation of SDA

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Accordingly, it is to be understood that the embodiments of the invention herein providing for an improved source of SDA for utilization in food products should not be limited to the specific examples. These examples are illustrative of the general applicability of the current invention to a vast range of food items. With the inclusion of SDA these items can be made with the same or better sensory qualities while significantly enhancing the nutritionally quality of the food produced for human consumption.

Moreover, the examples provided herein are merely illustrative of the application of the principles of the invention. It will be evident from the foregoing description that changes in the form, methods of use, and applications of the elements of the disclosed plant-derived could be used for applications not directly related to human consumption. Included in this field is the use of plant-derived SDA for the development of nutritionally enhanced feed for use in animal production industries generally including but not limited to: beef production; poultry production; pork production; and or, aquaculture. These variant uses may be resorted to without departing from the spirit of the invention, or the scope of the appended claims.

### LITERATURE CITED AND INCORPORATED BY REFERENCE

These references are specifically incorporated by reference relevant to the supplemental procedural or other details that they provide.
1. Cohen J. T., et al., A Quantitative Risk-Benefit Analysis Of Changes In Population Fish Consumption. AM J PREV MED. (2005) November; 29(4):325-34.
2. Codex Standards For Edible Fats And Oils, in CODEX ALIMENTARIUS COMMISSION. (Supplement 1 to Codex Alimentarius)(Volume XI, Rome, FAO/WHO(1983)).
3. Report of the Fourteenth Session of the Codex Committee on Fats and Oils, London, 27 September-1 Oct. 1993, CODEX ALIMENTARIUS COMMISSION. (Alinorm 95/17. Rome, FAO/WHO(1993)).
4. DICTIONARY OF FOOD SCIENCE AND TECHNOLOGY, p 141, 151 (Blackwell publ.)(Oxford UK, 2005).
5. Finley, J. W., OMEGA-3 FATTY ACIDS: CHEMISTRY, NUTRITION, AND HEALTH EFFECTS, (ed. John W. Finley) (Publ. American Chemical Society, Wash. DC.)(ACS Symposium, May 2001)(Series Volume: 105-37788).
6. Gebauer S. K., et al., N-3 Fatty Acid Dietary Recommendations And Food Sources To Achieve Essentiality And Cardiovascular Benefits, AM J CLIN NUTR. (2006) June; 83(6 Suppl):1526S-1535S.
7. Gelvin et al., PLANT MOLECULAR BIOLOGY MANUAL, (Kluwer Academic Publ. (1990)).
8. Gomez, M. L. M., et al., Sensory Evaluation of Sherry Vinegar: Traditional Compared to Accelerated Aging with Oak Chips, J. FOOD SCIENCE 71(3) S238-S242 (2006).
9. Guichardant M., et al., Stearidonic Acid, an Inhibitor of the 5-Lipoxygenase Pathway, A Comparison With Timnodonic And Dihomogammalinolenic Acid. LIPIDS. (1993) April; 28(4):321-24.
10. Gunstone, F. D., and Herslof, B. G. in, LIPID GLOSSARY 2, (Publ. The Oily Press Lipid Library, (2000), 250 pages).
11. Hersleth M., et al., Perception of Bread: A Comparison of Consumers and Trained Assessors, J. FOOD SCIENCE 70(2) S95-101 (2005).
12. James M. J., et al., Metabolism of Stearidonic Acid In Human Subjects: Comparison With The Metabolism of Other N-3 Fatty Acids. AM J CLIN NUTR. 2003 May;77(5): 1140-45.
13. Kindle, K., et al., PNAS, USA 87:1228, (1990).
14. Kitamura and Keisuke, Breeding Trials For Improving The Food-Processing Quality Of Soybeans, TRENDS FOOD SCI. & TECHNOL. 4:64-67 (1993).
15. La Guardia M., et al., Omega 3 Fatty Acids: Biological Activity And Effects On Human Health, PANMINERVA MED. 2005 December;47(4):245-57.
16. Liu, J., et al., Sensory and Chemical Analyses of Oyster Mushrooms (Pleurotus Sajor-Caju) Harvested from Different Substrates, J. FOOD SCIENCE 70(9): S586-S592 (2005).
17. MANUAL ON DESCRIPTIVE ANALYSIS TESTING, FOR SENSORY EVALUATION, (edit. Hootman, R. C., 1992) ASTM Manual Series: MNL 13 pp 1-51 (publ. ASTM).
18. Matta, Z., et al., Consumer and Descriptive Sensory Analysis ofBlack Walnut Syrup, J. FOOD SCIENCE 70(9): S610-S613 (2005).
19. Morrissey M. T., The Good, The Bad, And The Ugly: Weighing The Risks And Benefits Of Seafood Consumption, NUTR HEALTH. 2006; 18(2): 193-7.
20. Myers, R. A. and Worm, B., Rapid World Wide Depletion of Predatory Fish Communities, NATURE 423: 280-83 (2003).
21. O'Brien R. D., FATS AND OILS, FORMULATING AND PROCESSING FOR APPLICATIONS, (publ. CRC Press)(2^{nd} edit. 2003)
22. Omega Pure, FOOD PRODUCT APPLICATIONS, Product Insert (2006).
23. Potrykus, I., ANN. REV. PLANT PHYSIOL. PLANT MOL. BIOLOGY, 42:205, (1991).
24. Rocha-Uribe, A., Physical and Oxidative Stability ofMayonnaise Enriched with Different Levels of n-3 Fatty Acids and stored at Different Temperatures, IFT ANNUAL MEETING July 12-16 (2004), Las Vegas, USA.
25. Sidel & Stone, Sensory Science: Methodology in, HANDBOOK OF FOOD SCIENCE, TECHNOLOGY AND ENGINEERING VOL. 2, pp. 57-3 through 57-24 (edit. Hui, Y. H., 2005).
26. SOYFOODS COOKBOOK, @ soyfoods.com/recipes. (2006).
27. STANDARD GUIDE FOR SENSORY EVALUATION METHODS TO DETERMINE THE SENSORY SHELF-LIFE OF CONSUMER PRODUCTS, (publ. ASTM Int'l) publication E2454-05; pp. 1-9 (2005).
28. Ursin, V. M., Modification Of Plant Lipids For Human Health:Development Of Functional Land-Based Omega-3 Fatty Acids Symposium: Improving Human Nutrition Through Genomics, Proteomics And Biotechnologies. J. NUTR. 133: 4271-74 (2003).
29. Whelan J. and Rust C., Innovative Dietary Sources of N-3 Fatty Acids, ANNU. REV. NUTR. 26: 75-103 (2006).
30. Weissbach and Weissbach, METHODS FOR PLANT MOLECULAR BIOLOGY, (Academic Press, (1989)).
31. Wojciech, K. et al., Possibilities of Fish Oil Application for Food Products Enrichment with Omega-3 PUFA, INT'L J. FOOD SCI. NUTR. 50:39-49 (1999).

### PATENTS AND PATENT APPLICATIONS CITED AND INCORPORATED BY REFERENCE

Patents:
Abbruzzese—2002 U.S. Pat. No. 6,387,883
Akashe et al.,—2006, U.S. Pat. No. 7,037,547
Barclay et al., 1999, U.S. Pat. No. 5,985,348
Barclay et al., 1997, U.S. Pat. No. 5,656,319
Barclay et al., 1994, U.S. Pat. No. 5,340,594
Dartey et al.,—2002, U.S. Pat. No. 6,399,137
Dartey et al.,—2000, U.S. Pat. No. 6,123,978
Knutzon et al., 2002 U.S. Pat. No. 6,459,018
Schroeder et al.,—1990, U.S. Pat. No. 4,913,921
Wintersdorffet al.,—1972, U.S. Pat. No. 3,676,157

Applications:
Fillatti J., et al., U.S. Patent Application Publication No. 2004/0107460A1, Jun. 3, 2004, Nucleic Acid Constructs and Methods for Producing Altered Seed Oil Compositions.
Myhre et al.,—U.S. Patent Application Publication No. 2003/0082275A1, May 1, 2003, Drinkable Omega-3 Preparation and Storage Stabilization.
Palmer et al.,—U.S. Patent Application Publication No. 2005/0181019A1, Aug. 18, 2005, Nutrition Bar.
Perlman et al.,—U.S. Patent Application Publication No. 2005/0244564A1, Nov. 3, 2005, Oxidative Stabilization of Omega-3 Fatty Acids in Low Linoleic Acid-Containing Peanut Butter.
Shiiba, et al., U.S. Patent Application Publication No. 2006/006888A1, Mar. 23, 2004, Acidic Oil-In-Water Emulsion Compositions.
Siew, et al., U.S. Patent Application Publication No. 2004/0224071A1, Nov. 11, 2004, Process for Obtaining an Oil Composition and the Oil Composition Obtained Therefrom.

1-101. (canceled)

102. A food product comprising stearidonic acid exhibiting extended shelf-life against flavor degradation wherein said stearidonic acid is derived from a transgenic plant.

103. The product of claim 102 wherein said extended shelf-life comprises at least 5% longer shelf life than a corresponding concentration of EPA.

104. The product of claim 102 further comprising at least about 5 ppm tocopherols.

105. The product of claim 102 wherein said stearidonic acid comprises from 0.1% to 80% of said food product.

106. The product of claim 103 further comprising soy protein.

107. The product of claim 103 wherein said feed product comprises less than about 40% LA.

108. The product of claim 102 wherein said stearidonic acid is part of an oil fraction from an oilseed plant, and wherein said oilseed plant fraction is comprised of from 2% to 50% of said oilseed plant oil after plant produced seed and/or fragment is crushed to release said oil fraction.

109. The product of claim 102 further comprising: a) a moisture containing ingredient; and, b) sufficient stabilizer to form an emulsion, such that said food product is a stable emulsion.

110. The product of claim 109 wherein said emulsion is of the oil-in-water type and wherein said aqueous phase comprises 10% to 80% by weight of said food product.

111. An animal feed product containing stearidonic acid exhibiting extended product life wherein the stearidonic acid is derived from a transgenic plant and wherein said feed product can be utilized as animal feed for livestock and/or aquaculture.

112. The feed product of claim 111 wherein said livestock is selected from the group consisting of cattle, swine, poultry, and chicken.

113. The feed product of claim 111 wherein said aquaculture animal is selected from the group consisting of salmon, trout, catfish, tilapia, crustacean, and mackerel.

114. The feed product of claim 111 further comprising at least about 5 ppm tocopherols.

115. The feed product of claim 111 wherein said stearidonic acid comprises from 0.1% to 80% of said feed product.

116. The feed product of claim 115 further comprising soy protein.

117. The feed product of claim 115 wherein said feed product comprises less than about 40% LA.

118. A food ingredient comprising a transgenic soybean oil, wherein said transgenic soybean oil comprises at least about 0.2% SDA and at most about 40% LA based on the total weight of fatty acids or derivatives thereof in the composition, and wherein said transgenic soybean oil comprises at least about 400 ppm tocopherols.

119. The food ingredient of claim 118 wherein said stearidonic acid comprises from 0.1% to 80% of said food ingredient.

120. The food ingredient of claim 118 wherein the transgenic soybean oil comprises at least one stabilizing agent selected from the group consisting of citric acid, t-butyl hydroquinone, ascorbyl palmitate, propyl gallate, and combinations thereof.

121. The food ingredient of claim 118 wherein said transgenic soybean oil further comprises of at least 10% SDA and at most about 35% LA based on the total weight of fatty acids or derivatives thereof in the composition.
